SEQUENCE LISTING (9 sequences)

SEQ ID NO: 1 - protein, 365 aa, Homo sapiens
MAKVAKDLNPGVKKMSLGQLQSARGVACLGCKGTCSGFEPHSWRKICKSCKCSQEDHCLTSDLEDDRKIGRLLMDSKYSTLTARVKGGDGIRIYKRNRMIMTNPIATGKDPTFDTITYEWAPPGVTQKLGLQYMELIPKEKQPVTGTEGAFYRRRQLMHQLPIYDQDPSRCRGLLENELKLMEEFVKQYKSEALGVGEVALPGQGGLPKEEGKQQEKPEGAETTAATTNGSLSDPSKEVEYVCELCKGAAPPDSPVVYSDRAGYNKQWHPTCFVCAKCSEPLVDLIYFWKDGAPWCGRHYCESLRPRCSGCDEIIFAEDYQRVEDLAWHRKHFVCEGCEQLLSGRAYIVTKGQLLCPTCSKSKRS

SEQ ID NO: 2 - protein, 292 aa, Homo sapiens
MDSKYSTLTARVKGGDGIRIYKRNRMIMTNPIATGKDPTFDTITYEWAPPGVTQKLGLQYMELIPKEKQPVTGTEGAFYRRRQLMHQLPIYDQDPSRCRGLLENELKLMEEFVKQYKSEALGVGEVALPGQGGLPKEEGKQQEKPEGAETTAATTNGSLSDPSKEVEYVCELCKGAAPPDSPVVYSDRAGYNKQWHPTCFVCAKCSEPLVDLIYFWKDGAPWCGRHYCESLRPRCSGCDEIIFAEDYQRVEDLAWHRKHFVCEGCEQLLSGRAYIVTKGQLLCPTCSKSKRS

SEQ ID NO: 3 - protein, 253 aa, Homo sapiens
MFGMQGDVFGLRATFMEGLQYMELIPKEKQPVTGTEGAFYRRRQLMHQLPIYDQDPSRCRGLLENELKLMEEFVKQYKSEALGVGEVALPGQGGLPKEEGKQQEKPEGAETTAATTNGSLSDPSKEVEYVCELCKGAAPPDSPVVYSDRAGYNKQWHPTCFVCAKCSEPLVDLIYFWKDGAPWCGRHYCESLRPRCSGCDEIIFAEDYQRVEDLAWHRKHFVCEGCEQLLSGRAYIVTKGQLLCPTCSKSKRS

SEQ ID NO: 4 - protein, 247 aa, Homo sapiens
MAKVAKDLNPGVKKMSLGQLQSARGVACLGCKGTCSGFEPHSWRKICKSCKCSQEDHCLTSDLEDDRKIGRLLMDSKYSTLTARVKGGDGIRIYKRNRMIMTNPIATGKDPTFDTITYEWAPPGVTQKLGLQYMELIPKEKQPVTGTEGAFYRRRQLMHQLPIYDQDPSRCRGLLENELKLMEEFVKQYKSEALGVGEVALPGQGGLPKEEGKQQEKPEGAETTAATTNGSLSDPSKEVEYKPVPLV

SEQ ID NO: 5 - DNA, 1757 bp, Homo sapiens
agctgcgcctggctgcgcacagagctccctcccaggcccgcgaacttggccattcagccgccgctgtccccgctgcgcgccctcgcgcctctgcctgagaagccaggcgctgttcccccaccccagaagaggatggcaaaggtggctaaggacctcaacccaggagttaaaaagatgtccctgggccagctgcagtcagcaagaggtgtggcatgtttgggatgcaaggggacgtgttcgggcttcgagccacattcatggaggaaaatatgcaagtcttgcaaatgcagccaagaggaccactgcctaacatctgacctagaagacgatcggaaaattggccgcttgctgatggactccaagtattccaccctcactgctcgggtgaaaggcggggacggcatccggatttacaagaggaaccggatgatcatgaccaaccctattgctactgggaaagatcccacttttgacaccatcacctacgagtgggctccccctggagtcacccagaaactgggactgcagtacatggagctcatccccaaggagaagcagccagtgacaggcacagagggtgccttttaccgccgccgccagctcatgcaccagctccccatctatgaccaggatccctcgcgctgccgtggacttttggagaatgagttgaaactgatggaagaatttgtcaagcaatataagagcgaggccctcggcgtgggagaagtggccctcccggggcagggtggcttgcccaaggaggaggggaagcagcaggaaaagccagagggggcagagaccactgctgctaccaccaacggcagtctcagtgacccgtccaaagaagtggaatacgtctgcgagctctgcaagggagcggcccctcctgacagccccgtggtctactcggacagggcaggctacaacaagcagtggcaccccacctgctttgtgtgtgccaagtgctccgagccgctggtggacctcatctacttctggaaggatggtgcaccctggtgcggccgccattactgcgagagtctgcggccccggtgctccggctgcgatgagataatattcgctgaggactaccagcgtgtggaagatctggcctggcaccgaaagcactttgtctgtgagggttgtgagcagctgctgagcggccgggcgtacatcgtcaccaagggtcagcttctgtgcccaacttgcagcaagtccaaacgctcctgaagggctgcccacccacagccagaatccacaggatcccaccgagaaggagagccaggtgtgccgagaccatcctaagggtccgatgtgacagcaagcaagtgaaataaacaatgatttgcttttcagtgagaatatatatatgagatatatatagatatatattctaggttgggtggtggtagatccttgagggtcagtagtttcaaaaccaaaaatattctaagaagtcttaggatggagttccttttctttctgttgttgtttcccagctacaaccaactaaagacacaaatggcgttctgcaaggggactctgggaggagttttccagaatgcaattccgagtgagcaaatcgcatagctgtagaatgtgcgtgcttttttgtggacacaggagctcctccaggagcaggctgggatcccaactatcgcttgttgcctctttttcaagtggaatttgaattttaaataaacaactttttttggcatgataaacagatcaataaaagttttgtgaattccacatac

SEQ ID NO: 6 - DNA, 1668 bp, Homo sapiens
agctgcgcctggctgcgcacagagctccctcccaggcccgcgaacttggccattcagccgccgctgtccccgctgcgcgccctcgcgcctctgcctgagaagccaggcgctgttcccccaccccagaagaggatggcaaaggtggctaaggacctcaacccaggagttaaaaaggaaaatatgcaagtcttgcaaatgcagccaagaggaccactgcctaacatctgacctagaagacgatcggaaaattggccgcttgctgatggactccaagtattccaccctcactgctcgggtgaaaggcggggacggcatccggatttacaagaggaaccggatgatcatgaccaaccctattgctactgggaaagatcccacttttgacaccatcacctacgagtgggctccccctggagtcacccagaaactgggactgcagtacatggagctcatccccaaggagaagcagccagtgacaggcacagagggtgccttttaccgccgccgccagctcatgcaccagctccccatctatgaccaggatccctcgcgctgccgtggacttttggagaatgagttgaaactgatggaagaatttgtcaagcaatataagagcgaggccctcggcgtgggagaagtggccctcccggggcagggtggcttgcccaaggaggaggggaagcagcaggaaaagccagagggggcagagaccactgctgctaccaccaacggcagtctcagtgacccgtccaaagaagtggaatacgtctgcgagctctgcaagggagcggcccctcctgacagccccgtggtctactcggacagggcaggctacaacaagcagtggcaccccacctgctttgtgtgtgccaagtgctccgagccgctggtggacctcatctacttctggaaggatggtgcaccctggtgcggccgccattactgcgagagtctgcggccccggtgctccggctgcgatgagataatattcgctgaggactaccagcgtgtggaagatctggcctggcaccgaaagcactttgtctgtgagggttgtgagcagctgctgagcggccgggcgtacatcgtcaccaagggtcagcttctgtgcccaacttgcagcaagtccaaacgctcctgaagggctgcccacccacagccagaatccacaggatcccaccgagaaggagagccaggtgtgccgagaccatcctaagggtccgatgtgacagcaagcaagtgaaataaacaatgatttgcttttcagtgagaatatatatatgagatatatatagatatatattctaggttgggtggtggtagatccttgagggtcagtagtttcaaaaccaaaaatattctaagaagtcttaggatggagttccttttctttctgttgttgtttcccagctacaaccaactaaagacacaaatggcgttctgcaaggggactctgggaggagttttccagaatgcaattccgagtgagcaaatcgcatagctgtagaatgtgcgtgcttttttgtggacacaggagctcctccaggagcaggctgggatcccaactatcgcttgttgcctctttttcaagtggaatttgaattttaaataaacaactttttttggcatgataaacagatcaataaaagttttgtgaattccacatac

SEQ ID NO: 7 - DNA, 1501 bp, Homo sapiens
agctgcgcctggctgcgcacagagctccctcccaggcccgcgaacttggccattcagccgccgctgtccccgctgcgcgccctcgcgcctctgcctgagaagccaggcgctgttcccccaccccagaagaggatggcaaaggtggctaaggacctcaacccaggagttaaaaagatgtccctgggccagctgcagtcagcaagaggtgtggcatgtttgggatgcaaggggacgtgttcgggcttcgagccacattcatggagggactgcagtacatggagctcatccccaaggagaagcagccagtgacaggcacagagggtgccttttaccgccgccgccagctcatgcaccagctccccatctatgaccaggatccctcgcgctgccgtggacttttggagaatgagttgaaactgatggaagaatttgtcaagcaatataagagcgaggccctcggcgtgggagaagtggccctcccggggcagggtggcttgcccaaggaggaggggaagcagcaggaaaagccagagggggcagagaccactgctgctaccaccaacggcagtctcagtgacccgtccaaagaagtggaatacgtctgcgagctctgcaagggagcggcccctcctgacagccccgtggtctactcggacagggcaggctacaacaagcagtggcaccccacctgctttgtgtgtgccaagtgctccgagccgctggtggacctcatctacttctggaaggatggtgcaccctggtgcggccgccattactgcgagagtctgcggccccggtgctccggctgcgatgagataatattcgctgaggactaccagcgtgtggaagatctggcctggcaccgaaagcactttgtctgtgagggttgtgagcagctgctgagcggccgggcgtacatcgtcaccaagggtcagcttctgtgcccaacttgcagcaagtccaaacgctcctgaagggctgcccacccacagccagaatccacaggatcccaccgagaaggagagccaggtgtgccgagaccatcctaagggtccgatgtgacagcaagcaagtgaaataaacaatgatttgcttttcagtgagaatatatatatgagatatatatagatatatattctaggttgggtggtggtagatccttgagggtcagtagtttcaaaaccaaaaatattctaagaagtcttaggatggagttccttttctttctgttgttgtttcccagctacaaccaactaaagacacaaatggcgttctgcaaggggactctgggaggagttttccagaatgcaattccgagtgagcaaatcgcatagctgtagaatgtgcgtgcttttttgtggacacaggagctcctccaggagcaggctgggatcccaactatcgcttgttgcctctttttcaagtggaatttgaattttaaataaacaactttttttggcatgataaacagatcaataaaagttttgtgaattccacatac

SEQ ID NO: 8 - DNA, 2261 bp, Homo sapiens
agctgcgcctggctgcgcacagagctccctcccaggcccgcgaacttggccattcagccgccgctgtccccgctgcgcgccctcgcgcctctgcctgagaagccaggcgctgttcccccaccccagaagaggatggcaaaggtggctaaggacctcaacccaggagttaaaaagatgtccctgggccagctgcagtcagcaagaggtgtggcatgtttgggatgcaaggggacgtgttcgggcttcgagccacattcatggaggaaaatatgcaagtcttgcaaatgcagccaagaggaccactgcctaacatctgacctagaagacgatcggaaaattggccgcttgctgatggactccaagtattccaccctcactgctcgggtgaaaggcggggacggcatccggatttacaagaggaaccggatgatcatgaccaaccctattgctactgggaaagatcccacttttgacaccatcacctacgagtgggctccccctggagtcacccagaaactgggactgcagtacatggagctcatccccaaggagaagcagccagtgacaggcacagagggtgccttttaccgccgccgccagctcatgcaccagctccccatctatgaccaggatccctcgcgctgccgtggacttttggagaatgagttgaaactgatggaagaatttgtcaagcaatataagagcgaggccctcggcgtgggagaagtggccctcccggggcagggtggcttgcccaaggaggaggggaagcagcaggaaaagccagagggggcagagaccactgctgctaccaccaacggcagtctcagtgacccgtccaaagaagtggaatacaagccagtcccactcgtgtgataacccattaatctattaagccataagtggattaatccattcctgaggacctgagccctcacgacccaatcatctcttaaaggccccacctctcaatactgccatgcagaggattatgtttcaacctgagtgtttggaggggatgttcaacccataggaagtggcagtgtggaagaagtgctgctgaggagtgagtcactgggggccattttgagaaaacagaaaggagaagccagagttggggagatgaaagcctcatggcttggtttgtcttaaactgccccacagaaggcgaaaggaatgcttgaggctggaccacgtgggtctagcgtgtactgcgtttctggtccccagcccctgttttaccttttgctcctcctgccccatcaaccaagtgtcttcatttgtttctatggcaattaacttttggagatagaagtcccagcacacgagatccccaagcacattatctaccttgctgaacaggctggcagtcacacatgagccaggcgacccagggaaatgccagcccaaacaaagctgctgccacatccagagagggccggactctttctcccttgtagtcactcaagctaatcatccaaaacctgcatcctccatctccaagccccatcttattagcaccatctgggattgccaaccaagaaactgttttatctgagaactctaagaccaaagaacaagatttatttcctctactacagatttggcagtgacgcataaaaggcccatttctcaggaagaatacatgtcctaaggatgtaaaaaagaaaaaaatattagatctagttaccatggtctataaactggtcttttcccgccccaccctgatcctggcttctgtccaccctcaaatagctgtttgttcataaaccctaaatactagataattctaagttggaaggagacctctaagtcactgtagcatttccaaatcgccattcccaagagacatgtggatctgacatcgtgttttattcttgactgagcctcgcatatttgttctgtgtggaacaaaggcaaaggcagcccaagaacccgggtccttgcctacagtcagctttaggaaatgattgtgaacttgggaagcatttaaatagcaatactagacagtaaatggaaaaggccaaagtcagaaaataagtagggattccaaaggaagcctttattggttgggctaggctgggctagctgtggaagatagacttctatgtccctgccccaaccacaattttactttaattattatgtaattagtgaatcgatgtctgtcaccgtctgtagatgctgaggtcttgttcatctctttatttgcattgatatacatagccattgctcaataaatatgtgacccatgaaaaaaa

SEQ ID NO: 9 - protein, 425 aa, Homo sapiens
MSSILPFTPPIVKRLLGWKKGEQNGQEEKWCEKAVKSLVKKLKKTGQLDELEKAITTQNVNTKCITIPRSLDGRLQVSHRKGLPHVIYCRLWRWPDLHSHHELRAMELCEFAFNMKKDEVCVNPYHYQRVETPVLPPVLVPRHTEIPAEFPPLDDYSHSIPENTNFPAGIEPQSNIPETPPPGYLSEDGETSDHQMNHSMDAGSPNLSPNPMSPAHNNLDLQPVTYCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSIRCSSVS

(19) United States
(12) Patent Application Publication
(10) Pub. No.: US 2019/0360054 A1
(43) Pub. Date: Nov. 28, 2019

(54) METHOD FOR DIAGNOSIS OF METASTATIC CANCER OR TAXANE-BASED DRUG-RESISTANT CANCER BY USING LMCD1 PROTEIN AND GENE CODING THEREFOR

(71) Applicants: AICT (ADVANCED INSTITUTES OF CONVERGENCE TECHNOLOGY), Gyeonggi-do (KR); THERAGEN ETEX CO., LTD, Gyeonggi-do (KR)

(72) Inventors: Seong Jin KIM, Gyeonggi-do (KR); Jin Ah PARK, Gyeonggi-do (KR); Eun Jin BAE, Seoul (KR)

(73) Assignees: AICT (ADVANCED INSTITUTES OF CONVERGENCE TECHNOLOGY), Gyeonggi-do (KR); THERAGEN ETEX CO., LTD, Gyeonggi-do (KR)

(21) Appl. No.: 16/413,014

(22) Filed: May 15, 2019

Related U.S. Application Data
(63) Continuation-in-part of application No. PCT/KR2016/013828, filed on Nov. 29, 2016.

(30) Foreign Application Priority Data
Nov. 15, 2016 (KR) 10-2016-0152231

(51) Int. Cl. C12Q 1/6886; G01N 33/50; A61P 35/04

(52) U.S. Cl. CPC C12Q 1/6886; C12Q 2600/118; A61P 35/04; G01N 33/5029

(57) ABSTRACT

The present disclosure relates to a method of diagnosing metastatic cancer or taxane-based drug-resistant cancer, including: measuring an LMCD1 expression level from a complex formed by bringing, into contact with a sample isolated from a subject, an antibody, peptide, protein, or combination thereof that specifically binds to an LMCD1 protein or a fragment thereof; or a probe, primer, nucleotide, or combination thereof that specifically binds to a nucleotide sequence encoding the LMCD1 protein; and comparing the measured LMCD1 expression level of the sample with a measured LMCD1 expression level of a control, and a kit for diagnosing metastatic cancer or taxane-based drug-resistant cancer, including a composition for diagnosing metastatic cancer or taxane-based drug-resistant cancer.

### RELATED APPLICATIONS

The present application is a continuation-in-part of PCT Application No. PCT/KR2016/013828 filed Nov. 29, 2016, which claims priority to Korean Application No. 10-2016-0152231, filed Nov. 15, 2016, all of which are hereby incorporated in their entirety by reference as set forth herein.

### TECHNICAL FIELD

The present disclosure relates to a method and composition for diagnosing metastatic cancer or taxane-based drug-resistant cancer by using the degree of Smad3 protein phosphorylation and LMCD1 induced thereby as biomarkers.

### REFERENCE TO SEQUENCE LISTING

The present application contains a Sequence Listing that was filed as an ASCII text file via EFS-Web on May 15, 2019. The ASCII text file is entitled “AIY0001IA-Sequence_Listings.txt,” was created on Nov. 15, 2016 and is 23,000 bytes in size.

### BACKGROUND ART

Smad3 is a vital messenger of the TGF-β1 signaling pathway and mediates cell function by regulating the expression of active genes by transmission of a signal activated through receptor phosphorylation to the nucleus. TGF-β1 acts, in a dual manner, as a tumor suppressor by regulating the proliferation of epithelial cells at the early stage of a tumor in the onset and progression of cancer, but also as a tumor promoter by enhancing epithelial mesenchymal migration and invasion by being greatly activated at the terminal stage of a tumor. Recent studies have shown that the TGF-β1 signaling pathway mediates the resistance of therapeutic agents targeting breast cancer, colorectal cancer, and the like and general anticancer therapeutic agents, and thus is essential for providing fundamental diagnoses and therapies in the treatment and recurrence of cancer.

In this regard, it is known that the function of Smad3 is regulated by activation of a phosphorylation site located in Smad3. The phosphorylation site of Smad3 is concentrated at the site of a linker via which two domains of the amino terminus and the carboxy terminus are linked to each other and at the carboxy terminus, and signal transduction by receptor phosphorylation is predominantly regulated by carboxy terminal phosphorylation. Recent studies also have shown that the TGF-β1-mediated signaling pathway regulates the production and metastasis of breast cancer cells, depending on a phosphorylation state at the linker of the Smad3 protein.

Meanwhile, paclitaxel, which is widely used clinically for the treatment of breast cancer, is an anticancer agent that kills cells by inhibiting polymerization of microtubules during cell mitosis, and is widely applied in the treatment of cancers including lung cancer, breast cancer, ovarian cancer, and gastric cancer due to its excellent anticancer effect. However, cancers that are resistant to paclitaxel regardless of chemotherapy stages have been reported, paclitaxel-resistant breast cancer patients frequently experience recurrence and metastasis, and it has been frequently reported that paclitaxel has side effects such as bone marrow lymphopenia, secondary infection, hypersensitivity, and the like in spite of its excellent anticancer effect. Thus, when paclitaxel is continuously administered to patients having resistance to paclitaxel, therapeutic prognosis is deteriorated and the appropriate treatment period is missed, which may have a great influence on the survival rate of patients.

### DESCRIPTION OF EMBODIMENTS

### Technical Problem

Provided is a method of diagnosing metastatic cancer or taxane-based drug-resistant cancer, including: measuring an LMCD1 expression level from a complex formed by bringing, into contact with a sample isolated from a subject, an antibody, a peptide, a protein, or a combination thereof that specifically binds to the LMCD1 protein or a fragment thereof; or a probe, a primer, a nucleotide, or a combination thereof that specifically binds to a nucleotide sequence encoding the LMCD1 protein; and comparing the measured LMCD1 expression amount of the sample with an LMCD1 expression level of a control.

Provided is a composition for diagnosing metastatic cancer or taxane-based drug-resistant cancer, including: an antibody, a peptide, a protein, or a combination thereof that specifically binds to the LMCD1 protein or a fragment thereof; or a probe, a primer, a nucleotide, or a combination thereof that specifically binds to a nucleotide sequence encoding the LMCD1 protein.

Provided is a kit for diagnosing metastatic cancer or taxane-based drug-resistant cancer, including the above-described composition and a reagent for detecting the composition.

### Solution to Problem

According to an aspect of the present disclosure, there is provided a method of diagnosing metastatic cancer or taxane-based drug-resistant cancer, including: measuring an LMCD1 expression level from a complex formed by bringing, into contact with a sample isolated from a subject, an antibody, a peptide, a protein, or a combination thereof that specifically binds to the LIM and Cysteine Rich Domains 1 (LMCD1) protein or a fragment thereof; or a probe, a primer, a nucleotide, or a combination thereof that specifically binds to a nucleotide sequence encoding the LMCD1 protein; and comparing the measured LMCD1 expression level of the sample with a measured LMCD1 expression level of a control.

In the present disclosure, the LMCD1 protein may be a protein derived from a human (Homo sapiens) or a mouse (Mus musculus), but the same protein may also be expressed in other mammals such as monkeys, cows, horses, and the like, and human-derived LMCD1 may have four or more isoforms. LMCD1 may be translated from mRNA including GenBank Accession No. NM_014583.3 (SEQ ID NO: 5), NM_001278233.1 (SEQ ID NO: 6), NM_001278234.1 (SEQ ID NO: 7), or NM_001278235.1 (SEQ ID NO: 8) into a peptide or protein having an amino acid sequence represented by NP_055398.1 (SEQ ID NO: 1), NP_001265162.1 (SEQ ID NO: 2), NP_001265163.1 (SEQ ID NO: 3), or NP_001265164.1 (SEQ ID NO: 4). Even though some nucleotide sequences or amino acid sequences are inconsistent with each other between the mRNA of SEQ ID NOS: 5 to 8 and the protein of SEQ ID NOS: 1 to 4, nucleotide sequences or amino acid sequences having the same biological activity may be regarded as LMCD1 mRNA or the LMCD1 protein. Thus, in one embodiment, the LMCD1 protein may include any one sequence selected from SEQ ID NOS: 1 to 4, and a nucleotide sequence encoding the LMCD1 protein may include any one sequence selected from SEQ ID NOS: 5 to 8.

The nucleotide encoding the LMCD1 protein may have a base sequence with at least 60% homology, for example, at least 70% homology, at least 80% homology, at least 90% homology, at least 95% homology, at least 99% homology, or 100% homology, to any one sequence selected from SEQ ID NOS: 5 to 8. In addition, the nucleotide encoding the LMCD1 protein may have any one base sequence selected from SEQ ID NOS: 5 to 8, in which at least one base, at least two bases, at least three bases, at least four bases, at least five bases, at least six bases, or at least seven bases are different.

The LMCD1 protein may have an amino acid sequence with at least 60% homology, for example, at least 70% homology, at least 80% homology, at least 90% homology, at least 95% homology, at least 99% homology, or 100% homology, to any one sequence selected from SEQ ID NOS: 1 to 4. In addition, the LMCD1 protein may have any one amino acid sequence selected from SEQ ID NOS: 5 to 8, in which at least one amino acid residue, at least two amino acid residues, at least three amino acid residues, at least four amino acid residues, at least five amino acid residues, at least six amino acid residues, or at least seven amino acid residues are modified.

The term “cancer” may be used interchangeably with the term “tumor,” and the cancer may be any one selected from the group consisting of cerebral spinal cord tumor, head and neck cancer, thymoma, multiple myeloma, acute leukemia, lung cancer, liver cancer, esophageal cancer, gastric cancer, large intestine cancer, small intestine cancer, pancreatic cancer, oral cancer, brain tumor, thyroid cancer, parathyroid cancer, kidney cancer, cervical cancer, sarcoma, prostate cancer, urethral cancer, bladder cancer, testicular cancer, blood cancer, lymphoma, skin cancer, psoriasis, and fibroadenoma. In one embodiment, the cancer is breast cancer.

The term “taxane-based drug” refers to a drug belonging to the taxane family, which is the diterpene family and may have the same meaning as the term “taxoid,” and the taxane-based drug is derived from a plant of the genus Taxus and has a taxadiene nucleus. In one embodiment, the taxane-based drug may be paclitaxel (or Taxol®), doxetaxel (Taxotere®), carbazitaxel, or a combination thereof.

The term “resistance” as used herein means that the prevention, alleviation, or treatment of cancer is not effectively implemented by administration of a taxane-based drug, and more particularly means that the efficacy of a taxane-based drug, which interferes with the function of microtubules by stabilizing GDP-conjugated tubules among microtubules essential for cell division, is reduced or not induced, and is intended to include not only cases in which efficacy is not induced at an initial chemotherapy stage, but also cases in which efficacy of a taxane-based drug is reduced or not induced by acquired resistance occurring during repeated treatment, the progression of diseases stages, or the treatment of cancer occurring in other organs.

The term “antibody” as used herein refers to a specific immunoglobulin directed against an antigenic site. The antibody refers to an antibody specifically binding to the LMCD1 protein, and the LMCD1 protein encoded by the LMCD1 gene may be produced by cloning the LMCD1 gene into an expression vector, and an antibody may be prepared from the produced LMCD1 protein using a method commonly used in the art. The antibody includes a polyclonal antibody and a monoclonal antibody, and all immunoglobulin antibodies are included. The antibody includes not only a complete form having two full-length light chains and two full-length heavy chains, but also functional fragments of antibody molecules having an antigen-binding function due to having a specific antigen-binding site (binding domain) directed against an antigenic site although the antibody does not have a complete form having two light chains and two heavy chains and an intact antibody structure.

In the measurement of the LMCD1 expression amount from a complex formed by bringing, into contact with a sample isolated from a subject, a probe, a primer, a nucleotide, or a combination thereof that specifically binds to a nucleotide sequence encoding the LMCD1 protein, since the nucleotide sequence of the LMCD1 gene is known, one of ordinary skill in the art may design a primer or probe that specifically binds to a sequence of the LMCD1 gene based on the above sequence by using a method commonly used in the art.

The term “probe” as used herein refers to a fragment of a nucleic acid such as RNA, DNA, or the like, which corresponds to several to hundreds of bases capable of specifically binding to a nucleotide such as mRNA or the like. The probe may be labeled with a radioactive element or the like to identify the presence or absence of specific mRNA and amount (expression level) thereof. The probe may be produced in the form of an oligonucleotide probe, a single-stranded DNA probe, a double-stranded DNA probe, an RNA probe, or the like. According to one embodiment of the present disclosure, hybridization may be performed using a probe complementary to mRNA of the LMCD1 gene, which is a biomarker for the diagnosis of metastatic cancer or taxane-based drug-resistant cancer, and the presence or absence or degree of metastasis or taxane-based drug resistance may be measured by measuring an mRNA expression level through the degree of hybridization. Selection of a suitable probe and hybridization conditions may be appropriately selected according to a technique known in the art.

The term “primer” as used herein refers to a short nucleic acid sequence having a free 3′-hydroxyl group that is capable of forming a base pair with a complementary template and acts as a starting point for template strand duplication. A primer may initiate DNA synthesis in the presence of a reagent for polymerization (i.e., DNA polymerase or reverse transcriptase) in a proper buffer solution at a proper temperature and 4 different nucleoside triphosphates. According to one embodiment of the present invention, a desired expression amount of the LMCD1 protein may be measured by PCR amplification using a primer set of mRNA of the marker LMCD1, thereby diagnosing whether it is metastatic cancer or taxane-based drug-resistant cancer. PCR conditions and the length of the primer set may be appropriately selected according to a technique known in the art.

The term “nucleotide” as used herein refers to deoxyribonucleotide or ribonucleotide, and unless otherwise indicated herein, the nucleotide may include analogs of natural nucleotides and analogs in which sugar or base moieties are modified.

The probe, the primer, or the nucleotide may be chemically synthesized using a phosphoramidite solid support synthesis method or other well-known methods. These nucleotide sequences may also be modified using other various methods known in the art. Examples of such modifications include methylation, capping, substitution with one or more homologues of natural nucleotide, and modification between nucleotides, for example, modification into an uncharged linker (e.g., methyl phosphonate, phosphotriester, phosphoroamidate, carbamate, and the like) or a charged linker (e.g., phosphorothioate, phosphorodithioate, and the like).

The probe, the primer, or the nucleotide may have a length of about 10 nucleotides to about 100 nucleotides (hereinafter, referred to as “nt”), about 10 nt to about 90 nt, about 10 nt to about 80 nt, about 10 nt to about 70 nt, about 10 nt to about 60 nt, about 10 nt to about 50 nt, about 10 nt to about 40 nt, about 10 nt to about 30 nt, about 10 nt to about 25 nt, about 20 nt to about 100 nt, about 30 nt to about 90 nt, about 40 nt to about 80 nt, about 50 nt to about 70 nt, about 20 nt to about 60 nt, about 20 nt to about 50 nt, about 30 nt to about 40 nt, about 20 nt to about 30 nt, or about 20 nt to about 25 nt.

An mRNA expression level of the LMCD1 protein or the LMCD1 gene may be measured from a sample collected by selecting a site with a high expression level depending on a specific cancer, stage of cancer progression, or a specific site (e.g., luminal or basal/TNBC) in a particular cancer.

The term “measurement” as used herein refers to detection and quantification of a specific protein or nucleotide present in a sample. In one embodiment, the measurement may be performed by quantification using at least one method selected from Western blotting, ELISA, radioimmunoassay, radioimmunodiffusion, Ouchterlony immunodiffusion, rocket immunoelectrophoresis, immunohistostaining, immunoprecipitation assay, complement fixation assay, FACS, and a protein chip; at least one method selected from RT-PCR, RNase protection assay (RPA), Northern blotting, and a DNA chip; or a combination thereof, depending on a complex specific to the LMCD1 protein used in the method or a nucleotide sequence encoding the LMCD1 protein.

In one embodiment, the method of diagnosing metastatic cancer or taxane-based drug-resistant cancer of the present disclosure may further, before the measurement of the LMCD1 expression level, include bringing TGF-β into contact with the sample isolated from a subject.

In the present disclosure, transforming growth factor beta (TGFβ) is a secreted protein in which three isoforms, i.e., TGFβ1, TGFβ2, and TGFβ3, are present. TGFβ is encoded as a large protein precursor, TGFβ1 contains 390 amino acids, and each of TGFβ2 and TGFβ3 contains 412 amino acids. TGFβ is an N-terminal signal peptide of 20 to 30 amino acids required for secretion from cells, and has a proregion called latency associated peptide (LAP) and a 112 to 114 amino acid C-terminal region that becomes a mature TGFβ molecule after its release from the proregion by protein cleavage. Throughout the present specification, TGFβ is used as a meaning including a TGFβ precursor and mature TGFβ. Examples of TGFβ may include a TGFβ1 precursor and mature TGFβ1. TGFβ may be a protein derived from a human (Homo sapiens) or a mouse (Mus musculus), or a protein derived from other mammals such as monkeys, cows, horses, and the like.

In one embodiment, the method of diagnosing metastatic cancer or taxane-based drug-resistant cancer of the present disclosure may further include measuring the degree of Smad3 protein phosphorylation from a complex formed by bringing, into contact with the sample, an antibody, a peptide, a protein, or a combination thereof that specifically binds to the Smad3 protein or a fragment thereof, and the phosphorylation may mean phosphorylation of a Smad3 linker or its carboxy terminus. The Smad3 linker may refer to a portion including an amino acid sequence consisting of amino acid residues 143-230 in an amino acid sequence of SEQ ID NO: 9. The Smad3 carboxy terminus may refer to a portion including an amino acid sequence consisting of amino acid residues 422-425 in the amino acid sequence of SEQ ID NO: 9. In another embodiment, the phosphorylation may refer to phosphorylation of at least one selected from 179, 204, 208, and 213 in the Smad3 linker, amino acid residues 422, 423 and 425 in the Smad3 carboxy-terminus of the amino acid sequence of SEQ ID NO: 9.

In the present disclosure, the Smad3 protein may be a protein derived from a human (Homo sapiens) or a mouse (Mus musculus), but the same protein may also be expressed in other mammals such as monkeys, cows, horses, and the like, and the Smad3 protein may have an amino acid sequence of SEQ ID NO: 9. The Smad3 protein may include an amino acid sequence with at least 60% homology, for example, at least 70% homology, at least 80% homology, at least 90% homology, at least 95% homology, at least 99% homology, or 100% homology, to the amino acid sequence of SEQ ID NO: 9. In addition, the Smad3 protein may be an amino acid sequence of SEQ ID NO: 9 in which at least one amino acid residue, at least two amino acid residues, at least three amino acid residues, at least four amino acid residues, at least five amino acid residues, at least six amino acid residues, or at least seven amino acid residues are modified.

The term “phosphorylation” as used herein refers to substitution of some of specific amino acid residues with a phosphate group, and the phosphorylation may be induced in a cell by, for example, a phosphorylating enzyme such as a kinase. Generally, among amino acid residues constituting a protein (or a polypeptide), threonine and serine may be phosphorylated.

In the diagnosis method of the present disclosure, the antibody, the peptide, the protein, or a combination thereof may be specific to all phosphate groups of the Smad3 protein, phosphorylated amino acid residues of the Smad3 protein, only a phosphate group of the Smad3 linker or its carboxy terminus, only a phosphorylated amino acid residue of the Smad3 linker or its carboxy terminus, only one of phosphorylated amino acid residues 179, 204, 208, 213, 422, 423 and 425, at least two of phosphorylated amino acid residues 179, 204, 208, 213, 422, 423 and 425, or only all phosphorylated amino acid residues 179, 204, 208, 213, 422, 423 and 425.

The measurement of phosphorylation of the present disclosure may be performed using an antibody specific to specific phosphorylated amino acid residues, by measuring the degree of radiation using a radiation-labeled phosphate group, using an LC-MS/MS sequencing method, or the like. For example, when the LC-MS/MS sequencing method is used, the degree of phosphorylation or phosphorylated amino acid residues may be analyzed by measuring a change in mass of specific amino acid residues, mass of phosphate groups, and the like while sequencing a peptide sequence. In addition, when a radiation-labeled phosphate group, a chemiluminescent enzyme, or a fluorescent material is used, the degree of Smad3 protein phosphorylation may be measured by measuring radiation exposure dose, luminescence intensity, or fluorescence intensity.

In another embodiment, the diagnosis method of the present disclosure may further include measuring an expression level of a cancer stemness marker from a complex formed by bringing, into contact with a sample isolated from a subject, an antibody, a peptide, a protein, or a combination thereof that specifically binds to the cancer stemness marker or a fragment thereof; or a probe, a primer, a nucleotide, or a combination thereof that specifically binds to a nucleotide sequence encoding the cancer stemness marker. The term “cancer stemness” as used herein may refer to renewal and differentiation through asymmetric division like stem cells, but tumor production due to an impaired ability to regulate division, unlike normal stem cells. In one embodiment, the stemness marker may be Oct4, Nanog, Sox2, CD44, CD24, ALDH1, CD326 (EpCAM), or a combination thereof.

In one embodiment, when the method of diagnosing taxane-based drug-resistant cancer includes measuring an LMCD1 expression level from a complex formed by bringing, into contact with a sample isolated from a subject, a probe, a primer, a nucleotide, or a combination thereof that specifically binds to a nucleotide sequence encoding the LMCD1 protein, the measurement of the expression level may be performed by RT-PCR, RPA, northern blotting, and a DNA CHIP. The analysis method may be appropriately performed using a technique known to those of ordinary skill in the art.

In addition, in one embodiment, when the method of diagnosing metastatic cancer or taxane-based drug-resistant cancer includes measuring an LMCD1 expression level from a complex formed by bringing, into contact with a sample isolated from a subject, an antibody, a peptide, a protein, or a combination thereof that specifically binds to the LMCD1 protein or a fragment thereof; or a probe, a primer, a nucleotide, or a combination thereof that specifically binds to a nucleotide sequence encoding the LMCD1 protein, the measurement of the expression level may be performed using at least one method selected from Western blotting, enzyme linked immunosorbent assay (ELISA), radioimmunoassay (RIA), radioimmunodiffusion, Ouchterlony immunodiffusion, rocket immunoelectrophoresis, immunohistostaining, immunoprecipitation assay, complement fixation assay, FACS, and a protein chip. The analysis method may be appropriately performed using a technique known to those of ordinary skill in the art.

The subject to be diagnosed of the present disclosure may be a mammal. The mammal may be a human, a mouse, a rat, a cow, a goat, a pig, a horse, sheep, a dog, a cat, or a combination thereof. The sample may include a cell, lipid precursor cell, adipocyte of a subject, or a cell derived therefrom. Therefore, the term “sample isolated from a subject” as used herein refers to a biological sample derived from a subject to be diagnosed and isolated from the subject. For example, the sample may be isolated from a mammal, which is a target subject, and may be a cell, an organ, a cell lysate, whole blood, blood, serum, plasma, lymph fluid, extracellular fluid, body fluid, urine, feces, tissue, bone marrow, saliva, sputum, cerebrospinal fluid, or a combination thereof.

In one embodiment, in the diagnosis method of the present disclosure, the cancer may be any one selected from the group consisting of cerebral spinal cord tumor, head and neck cancer, thymoma, multiple myeloma, acute leukemia, lung cancer, liver cancer, esophageal cancer, gastric cancer, large intestine cancer, small intestine cancer, pancreatic cancer, oral cancer, brain tumor, thyroid cancer, parathyroid cancer, kidney cancer, cervical cancer, sarcoma, prostate cancer, urethral cancer, bladder cancer, testicular cancer, blood cancer, lymphoma, skin cancer, psoriasis, and fibroadenoma. In one embodiment, the caner may be breast cancer, and the breast cancer may be metastatic.

In one embodiment, the diagnosis method of the present disclosure may further include administering anti-cancer drug except taxane-based drug to the subject diagnosed with metastatic cancer or taxane-based drug-resistant cancer.

In one embodiment, the anti-cancer drug except taxane-based drug may be Anthracyclines (ex. Doxorubicin, Epirubicin), Platinum-based drugs (ex. Cisplatin, Carboplatin), Vinorelbine (ex. Navelbine), Capecitabine (ex. Xeloda), Gemcitabine (ex. Gemza), Ixabepilone (ex. Ixempra), Eribulin (ex. Halaven) or combination thereof.

In one embodiment, the anti-cancer drug except taxane-base drug may be Platinum-based drug. In one embodiment, it has been confirmed that LMCD1 does not induce platinum-based drug resistance, particularly cisplatin resistance.

According to another aspect of the present disclosure, there is provided a composition for diagnosing metastatic cancer or taxane-based drug-resistant cancer, including: an antibody, a peptide, a protein, or a combination thereof that specifically binds to the LMCD1 protein or a fragment thereof; or a probe, a primer, a nucleotide, or a combination thereof that specifically binds to a nucleotide sequence encoding the LMCD1 protein.

In one embodiment, the composition for diagnosing metastatic cancer or taxane-based drug-resistant cancer of the present disclosure may further include TGF-β.

In one embodiment, the composition for diagnosing metastatic cancer or taxane-based drug-resistant cancer of the present disclosure may also further include an antibody, a peptide, a protein, or a combination thereof that specifically binds to a phosphorylated Smad3 linker, Smad3 carboxy terminus, or a fragment thereof.

In one embodiment, the composition for diagnosing metastatic cancer or taxane-based drug-resistant cancer of the present disclosure may also further include: an antibody, a peptide, a protein, or a combination thereof that specifically binds to a cancer stemness marker protein or a fragment thereof in the sample isolated from a subject; or a probe, a primer, a nucleotide, or a combination thereof that specifically binds to a nucleotide sequence encoding the cancer stemness marker protein, and the stemness marker may be Oct4, Nanog, Sox2, CD44, CD24, ALDH1, CD326 (EpCAM), or a combination thereof.

The composition for diagnosing metastatic cancer or taxane-based drug-resistant cancer includes the above-described components required for performing the diagnosis method of the present disclosure, and may include not only a material capable of measuring an expression level of the above-described target factor (e.g., LMCD1, Oct4, Nanog, Sox2, CD44, CD24, ALDH1, CD326 (EpCAM), Smad3, or phosphorylated Smad3), i.e., an antibody, antigen-binding fragment, polypeptide, or protein that specifically binds to the above factor, a primer, probe, nucleotide, or combination thereof that specifically binds to a gene encoding the protein, but also one or more other constitutional compositions, solutions, or apparatuses that are suitable for an analysis method of measuring the expression level of the factor by using the composition.

According to another aspect of the present disclosure, there is provided a kit for diagnosing metastatic cancer or taxane-based drug-resistant cancer, including any one of the compositions of the present disclosure and a reagent for detecting the composition.

The kit for diagnosing metastatic cancer or taxane-based drug-resistant cancer may diagnose whether it is taxane-based drug-resistant cancer or metastatic cancer, by measuring an expression level of the LMCD1 protein through measurement of an mRNA or protein expression level of the LMCD1 gene. The kit for diagnosing taxane-based drug-resistant cancer may include not only the above-described material capable of measuring an expression level of the LMCD1 protein, i.e., an antibody, antigen-binding fragment, polypeptide, or protein that specifically binds to the LMCD1 protein, a primer, probe, nucleotide, or combination thereof that specifically binds to a gene encoding the LMCD1 protein, but also one or more other constitutional compositions, solutions, or apparatuses that are suitable for an analysis method of measuring the expression level of the LMCD1 protein by using the kit. In addition, the kit may further include: TGF-β, an antibody, peptide, protein, or combination thereof that specifically binds to a phosphorylated Smad3 protein (particularly, a phosphorylated Smad3 linker or its carboxy terminus) or a fragment thereof; or an antibody, peptide, protein, or combination thereof that specifically binds to a stemness marker protein or a fragment thereof, or a probe, primer, nucleotide, or combination thereof that specifically binds to a nucleotide sequence encoding the stemness marker protein, and may include one or more other constitutional compositions, solutions, or apparatuses that are suitable for an analysis method of measuring Smad3 protein phosphorylation or an expression level of the stemness marker protein. In addition, the kit may include one or more other constitutional compositions, solutions, or apparatuses that are suitable for an analysis method of measuring an expression level of the cancer stemness marker protein from a complex formed by bringing, into contact with a sample isolated from a subject, an antibody, peptide, protein, or combination thereof that specifically binds to a cancer stemness marker protein or a fragment thereof; or a probe, primer, nucleotide, or combination thereof that specifically binds to a nucleotide sequence encoding the cancer stemness marker protein.

According to one embodiment, when the kit for diagnosing metastatic cancer or taxane-based drug-resistant cancer of the present disclosure is a kit for measuring an mRNA expression level of the LMCD1 protein, the kit may be a kit including an essential element required for performing RT-PCR. An RT-PCR may include, in addition to a pair of primers specific to mRNA of each marker gene, a test tube or other suitable containers, a reaction buffer, deoxyribonucleotides (dNTPs), an enzyme such as Taq-polymerase and reverse transcriptase, a DNase inhibitor, an RNase inhibitor, dEPC-water, sterile water, and the like. In addition, the RT-PCR kit may include a pair of primers specific to a gene used as a quantitative control.

According to another embodiment, the kit for diagnosing metastatic cancer or taxane-based drug-resistant cancer of the present disclosure may include a substrate, a suitable buffer, a secondary antibody labeled with a chromogenic enzyme or a fluorescent material, and a chromogenic substrate to perform immunological detection of an antibody, antigen-binding fragment, polypeptide, or protein antibody that specifically binds to LMCD1, Oct4, Nanog, Sox2, CD44, CD24, ALDH1, CD326 (EpCAM), Smad3, or a phosphorylated Smad3 protein. The substrate may be a nitrocellulose membrane, a 96-well plate synthesized from polyvinyl resin, a 96-well plate synthesized from polystyrene resin, a glass slide, or the like, the chromogenic enzyme may be peroxidase or alkaline phosphatase, the fluorescent material may be FITC, RITC, or the like, and the chromogenic substrate may be 2,2′-azino-bis(3-ethylbenzothiazoline-6-sulfonic acid)(ABTS), o-phenylenediamine (OPD), tetramethyl benzidine (TMB), or the like.

According to another embodiment, the kit of the present disclosure may be a microarray for diagnosing metastatic cancer or taxane-based drug-resistant cancer, which is capable of measuring an mRNA expression level of a protein or a gene encoding the protein. The microarray for diagnosing metastatic cancer or taxane-based drug-resistant cancer may be readily manufactured by one of ordinary skill in the art using a method known in the art by using the marker of the present disclosure. According to one embodiment, the microarray may be a microarray in which mRNA of a gene encoding the LMCD1 protein, the Oct4 protein, the Nanog protein, the Sox2 protein, the CD44 protein, the CD24 protein, the ALDH1 protein, or the CD326 (EpCAM) protein, or cDNA of a sequence corresponding to a fragment thereof is attached as a probe to a substrate.

### Advantageous Effects of Disclosure

By using a method of diagnosing metastatic cancer or taxane-based drug-resistant cancer, according to an embodiment, which includes: measuring an LMCD1expression level from a complex formed by bringing, into contact with a sample isolated from a subject, an antibody, peptide, protein, or combination thereof that specifically binds to the LMCD1 protein or a fragment thereof; or a probe, primer, nucleotide, or combination thereof that specifically binds to a nucleotide sequence encoding the LMCD1 protein; and comparing the measured LMCD1 expression level of the sample with a measured LMCD1 expression level of a control, a subject having resistance to metastatic cancer or a taxane-based drug may be selected, and thus unnecessary drug treatment may be omitted.

By using a kit for diagnosing metastatic cancer or taxane-based drug-resistant cancer, according to another embodiment, including a composition for diagnosing metastatic cancer or taxane-based drug-resistant cancer, which includes an antibody, peptide, protein, or combination thereof that specifically binds to the LMCD1 protein or a fragment thereof; or a probe, primer, nucleotide, or combination thereof that specifically binds to a nucleotide sequence encoding the LMCD1 protein, and a reagent for detecting the composition, a subject with metastatic cancer or having resistance to a taxane-based drug may be readily and conveniently selected, and thus cancer therapeutic efficiency may be enhanced and rapid diagnosis and prognosis prediction are possible.

### BRIEF DESCRIPTION OF DRAWINGS

FIGS. 1A and 1B illustrate MTT analysis results of evaluation of the degree of susceptibility to a taxane-based anticancer agent in LMCD1-overexpressing metastatic breast cancer cells, and FIG. 1C illustrates MTT analysis results of evaluation of the degree of susceptibility to a taxane-based anticancer agent according to LMCD1 overexpression in metastatic breast cancer cells.

FIG. 2A illustrates RT-PCR results showing the degree of LMCD1 expression in metastatic breast cancer cells having resistance to a taxane-based anticancer agent, FIG. 2B illustrates MTT analysis results showing a change in susceptibility to a taxane-based anticancer agent according to the presence or absence of LMCD1 expression, and FIGS. 2C and 2D illustrate immunofluorescence results showing the degree of LMCD1 expression in a subject having resistance to a taxane-based anticancer agent.

FIGS. 3A-3C illustrate phosphorylated amino acid residues according to a phosphorylation site of the Smad3 protein.

FIGS. 4A and 4B illustrate MTT analysis results of resistance to a taxane-based anticancer agent after metastatic breast cancer cells were survived from cellular apoptosis mediated by infection with an Smad3 EPSM adenovirus, FIG. 4C illustrates RT-PCR results of measuring the degree of LMCD1 mRNA expression after metastatic breast cancer cells were survived from cellular apoptosis mediated by infection with an Smad3 EPSM adenovirus, FIGS. 4D and 4E illustrate MTT analysis results of evaluation of the degree of resistance to a taxane-based anticancer agent after metastatic breast cancer cells were infected with adenovirus inducing phosphorylation at different Smad3 phosphorylation sites, and FIG. 4F illustrates MTT analysis results of evaluation of a change in susceptibility to a taxane-based anticancer agent according to LMCD1 depletion after metastatic breast cancer cells were survived from cellular apoptosis mediated by infection with an Smad3 EPSM adenovirus.

FIG. 5A illustrates results of analyzing wound healing of an LMCD1-overexpressing model in metastatic breast cancer cells, FIGS. 5B and 5C illustrate an increase in migration and invasiveness according to LMCD1 overexpression by staining of nuclei of cells having passed through Transwell and Matrigel, FIGS. 5D and 5E illustrate migration inhibition phenomena when LMCD1 was depleted in metastatic breast cancer cells, by staining nuclei of cells having passed through Transwell, FIG. 5F illustrates RT-PCR results of labeled factors when LMCD1 was overexpressed in MCF10CA1a.cl1 metastatic breast cancer cells, FIG. 5G illustrates public data analysis results according to Oncomine Compendium of Expression Array data, and FIG. 5H illustrates immunofluorescent staining results showing the degree of LMCD1 expression in metastasized lung tissues after metastatic breast cancer cells were injected into a mouse lung metastasis model.

FIGS. 6A and 6B are a set of images showing analysis results of the degree of mammosphere formation after LMCD1 was overexpressed in metastatic breast cancer cells, and FIG. 6C illustrates RT-PCR results of measuring a change in mRNA of a stemness marker after LMCD1 was overexpressed in metastatic breast cancer cells.

FIGS. 7A and 7B illustrate microarray analysis results according to TGF-β treatment after metastatic breast cancer cells were infected with Smad3 wild-type and EPSM adenovirus, FIG. 7C illustrates genes exhibiting significant changes in a TGF-6 treated group after metastatic breast cancer cells were infected with Smad3 EPSM adenovirus, FIG. 7D illustrates results of measuring LMCD1 promoter activity according to TGF-β treatment after metastatic breast cancer cells were infected with Smad3 EPSM adenovirus, FIG. 7E illustrates results of measuring LMCD1 mRNA expression levels according to treatment with TGF-β and SB431542, which is a TGF-β inhibitor, after metastatic breast cancer cells were infected with Smad3 EPSM adenovirus, and FIG. 7F illustrates results of measuring LMCD1 expression levels by TGF-β treatment according to phosphorylation inhibition of each Smad3 phosphorylation site.

FIG. 8A illustrates results of identifying LMCD1 secretion in the conditioned media of metastatic breast cancer cells, FIG. 8B illustrates results of analyzing the interaction between LMCD1 and Annexin-II in metastatic breast cancer cells, FIG. 8C illustrates results of measuring LMCD1 secretion in the absence or presence of TGF-β after metastatic breast cancer cells were infected with Smad3 EPSM adenovirus, FIG. 8D illustrates results of measuring the activity of MMP-2 and MMP-9 in metastatic breast cancer cells according to phosphorylation status of each Smad3 phosphorylation site, FIGS. 8E to 8G illustrate an increase in invasiveness by staining of nuclei of cells having passed through Matrigel and in resistance to a taxane-based anticancer agent in benign breast cancer cells according to the treatment of LMCD1-containing conditioned media, FIGS. 8H to 8J illustrate an increase in invasiveness by staining of nuclei of cells having passed through Matrigel and in resistance to a taxane-based anticancer agent in metastatic breast cancer cells according to the treatment of LMCD1-containing conditioned media.

FIG. 9A illustrates ELISA analysis results of measuring LMCD1 protein level in the serums of healthy volunteers and breast cancer patients, FIG. 9B illustrates ELISA analysis results of measuring LMCD1 protein level in the serums of different subtypes of breast cancer patients.

### MODE OF DISCLOSURE

Hereinafter, the present disclosure will be described in further detail with reference to the following examples. However, these examples are provided for illustrative purposes only and are not intended to limit the scope of the present disclosure.

### Example 1: Verification of Resistance to Taxane-Based Anticancer Agent in LMCD1-Overexpressing Cells Through MTT Assay

To investigate whether LMCD1 protein-overexpressing breast cancer cells have resistance to a taxane-based anticancer agent, an MTT assay, which is used to evaluate cell viability, was performed on MCF-10CA1a.cl1, which are human MCF-10A-based breast cancer cell lines, and MDA-MB231. The MCF-10CA1a.cl1 and MDA-MB231 cell lines are characterized as malignant breast cancer cell lines with high metastasis.

In particular, human MCF-10A-based breast cancer cell lines, i.e., MCF-10CA1a.cl1 and MDA-MB231, were maintained in a DMEM culture solution containing 10% fetal bovine serum and 1% penicillin/streptomycin (WeIGENE) in a CO₂ incubator at 37° C.

To overexpress the LMCD1 protein in cells, LMCD1 was cloned into retroviral vector pLPCX, and LMCD1 retrovirus was obtained using Plat-GP cells. The metastatic breast cancer cells were infected with the LMCD1 retrovirus, LMCD1 protein-overexpressing cells were selected using puromycin, and then the selected cells were incubated in a DMEM culture solution containing 10% fetal bovine serum and 1% penicillin/streptomycin (WeIGENE) in a CO₂ incubator at 37° C.

The cells were seeded in 96 wells at a density of 3,000 cells per well and incubated for 24 hours. The culture solution was removed and a culture solution containing paclitaxel or docetaxel, which is a taxane-based anticancer agent, was added to each well to a final volume of 100 μl/well. After incubation for 72 hours, the culture solution was removed from the culture, and 0.5 mg/ml of 3-(4,5-dimethylthiazol-2-yl)-2,5-diphenyltetrazolium bromide (MTT) was added to each well, followed by incubation for 2 hours. Media containing an MTT solution was carefully removed, and 200 μl of dimethyl sulfoxide was added to each well, followed by incubation at room temperature for 5 minutes. Absorbance at 580 nm was measured using a 96-well microplate detector (Molecular Devices).

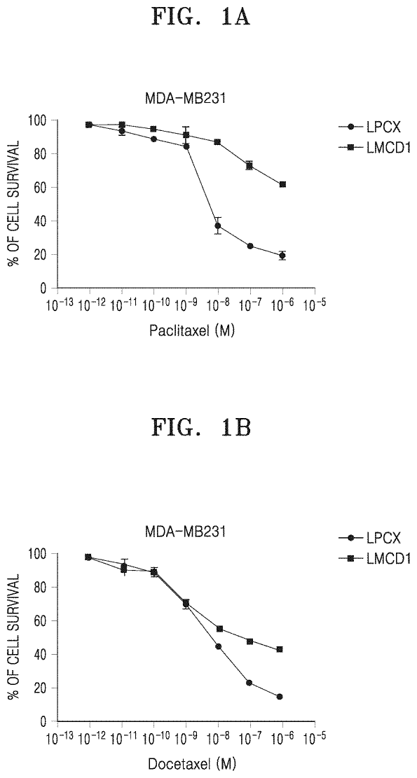

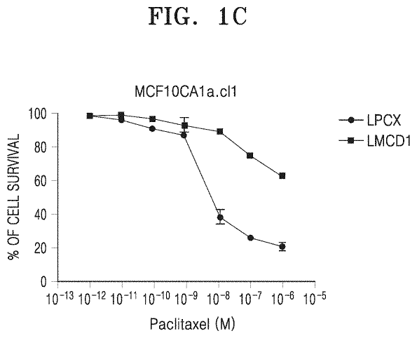

As a result, as illustrated in FIGS. 1A and 1B, it was confirmed that the LMCD1 protein-overexpressing MDA-MB231 cells exhibited resistance to the taxane-based anticancer agents. In addition, as illustrated in FIG. 1C, it was confirmed that resistance to paclitaxel was increased in the MCF10CA1a.cl1 cells when LMCD1 was overexpressed.

### Example 2: Evaluation of LMCD1 Overexpression in Cells with Resistance to Taxane-Based Anticancer Agent

2-1. Production of Cells with Resistance to Taxane-Based Anticancer Agent

To evaluate whether the LMCD1 protein is overexpressed in cells having resistance to a taxane-based anticancer agent, cells having resistance to a taxane-based anticancer agent were produced.

In particular, parental cells were treated with paclitaxel at a small concentration of 0.5 nM, and alive cells were repeated treated with paclitaxel three or four times while being passaged. After the repeated paclitaxel treatment processes, alive cells were treated with paclitaxel with an increment of 0.5 nM, and then passaging of alive cells was further repeated, thereby producing cells having resistance to paclitaxel that did not die even at an IC₅₀ value of 50 nM.

2.2. Evaluation of LMCD1 Expression Level in Cells with Resistance to Taxane-Based Anticancer Agent

An expression level of LMCD1 in taxane-based anticancer agent-resistant cells was evaluated at an mRNA level through RT-PCR, and it was evaluated whether susceptibility to the taxane-based anticancer agent was restored when the LMCD1 expression level was reduced by inhibiting LMCD1 expression.

In particular, total RNA was extracted from MCF10CA1a.cl1 cells (M4) and the paclitaxel-resistant MCF10CA1a.cl1 cells (M4 PTX) prepared according to Example 2-1 using a TRIzol reagent (Invitrogen) in accordance with the manufacturer's protocol. The total RNA was converted into cDNA using M-MLV reverse transcriptase (Promega). The cDNA was synthesized and subjected to RT-PCR using a specific primer pair by using an AccuPower™ PCR PreMix kit (Bioneer Co.). Gene mRNAs were normalized to GAPDH.

In addition, an MTT assay was performed to confirm whether susceptibility to a taxane-based anticancer agent is restored according to a decrease in LMCD1 expression level, in terms of cell viability. In particular, the paclitaxel-resistant MCF10CA1a.cl1 cells produced according to Example 2-1. The shRNA against LMCD1 was transfected into early-passage 293T cells, and then a lentivirus for LMCD1 depletion was produced. The paclitaxel-resistant MCF10CA1a.cl1 cells were infected with the lentivirus and then selected using puromycin, thereby producing LMCD1-depleted paclitaxel-resistant MCF10CA1a.cl1 cells (PTX-shLmcd1 #1).

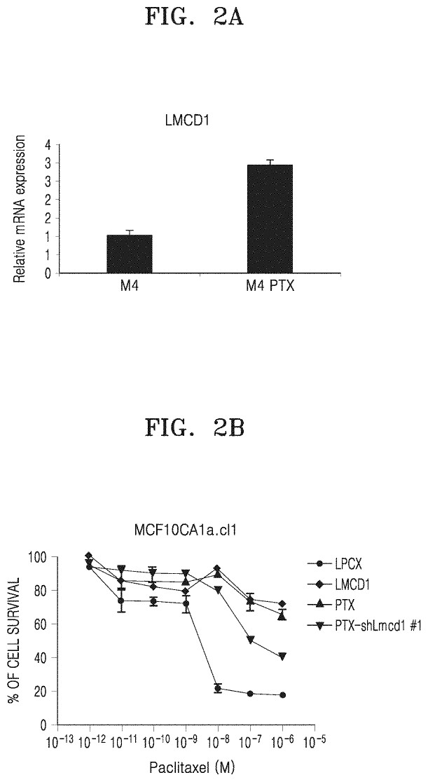

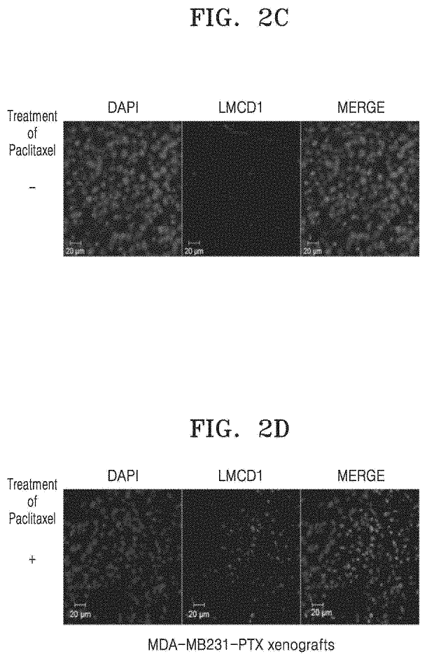

As a result, as illustrated in FIG. 2A, it was confirmed that the LMCD1 mRNA expression level was increased in the paclitaxel-resistant cells. Also in terms of cell viability, as illustrated in FIG. 2B, susceptibility to a taxane-based drug was reduced in LMCD1-overexpressing or paclitaxel-resistant cells (PTX) compared to a control (LPCX), thus exhibiting comparatively high viability, while being increased again when LMCD1 was depleted in the paclitaxel-resistant cells (PTX) (PTX-shLmcd1 #1).

2-3. Evaluation of LMCD1 Expression Level in Taxane-Based Anticancer Agent-Resistant Cells In Vivo

Animal experiments were conducted to investigate whether LMCD1 expression levels were also increased in a subject with resistance to a taxane-based anticancer agent.

In particular, female NOD/SCID mice were purchased from Orient Bio Inc. (Seongnam, S. Korea), and 2×10⁶ of paclitaxel-resistant MDA-MB231 cells were transplanted into the left fourth mammary fat pad of each female NOD/SCID mouse to prepare a xenograft model, and 10 weeks after transplantation, the mice were sacrificed for further analysis. All procedures were carried out in accordance with the guidelines provided by the CHA Hospital Animal Care and Use Committee. Xenograft paraffin blocks were prepared and cut into 4-μm-thickness sections, and paraffin was removed therefrom in xylene and alcohol. After blocking endogenous peroxidase activity and a retrieving antigen, the sections were blocked with PBS containing 5% BSA at 37° C. for 1 hour. The sections were incubated with anti-LMCD1 (Abcam) overnight at 4° C. Incubation was performed at 1:500 for 1 hour using Alexa Fluor 488-conjugated goat anti-rabbit IgG as a secondary antibody. The sections were placed in a culture solution containing DAPI (Vector Laboratories) and evaluated using a confocal laser scanning microscope (LSM-510; Carl Zeiss).

As a result, as illustrated in FIGS. 2C and 2D, it was confirmed that LMCD1 expression was increased in tumors derived from paclitaxel-resistant cells, as compared to a control.

### Example 3: Verification of LMCD1 Overexpression and the Effect of Different Smad3 Protein Phosphorylation in Paclitaxel-Resistant Cells

3-1. Production of Smad3 Phosphorylation Inhibition Model

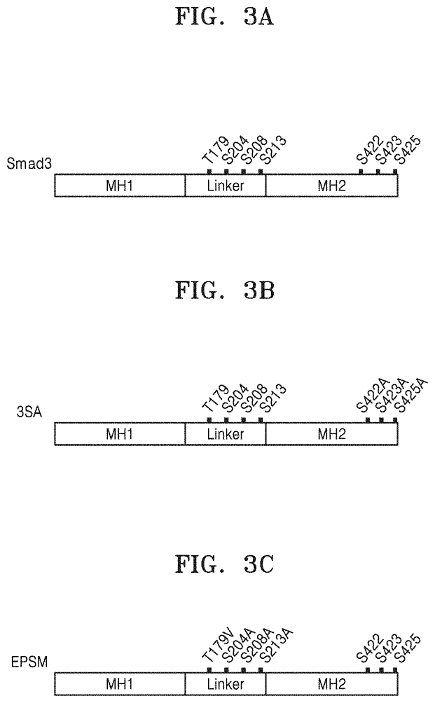

To verify whether LMCD1 overexpression and Smad3 linker phosphorylation inhibition are exhibited in paclitaxel-resistant cells, as illustrated in FIGS. 3A-3C, phosphorylation inhibition models according to Smad3 phosphorylation site were produced. GFP, Smad3 wild-type, C-terminal phosphorylation-inhibiting mutant (3SA), Smad3 linker phosphorylation-activating mutant (STD), and a Smad3 linker phosphorylation-inhibiting mutant (EPSM) adenovirus were supplied from Dr. Sushil G. Rane (NIDDK, NIH, Bethesda, Md.). To measure transduction efficiency, GFP adenovirus was used as a control. The cells were transfected with 75 to 100 virus particles/cell in a cell solution, and each transfection procedure was repeated three times.

3-2. Evaluation of LMCD1 Expression Level and Smad3 Protein Phosphorylation in Paclitaxel-Resistant Cells

To confirm whether LMCD1 overexpression and Smad3 linker phosphorylation inhibition occur in paclitaxel-resistant cells, resistance to paclitaxel according to a Smad3 phosphorylation site was evaluated in terms of LMCD1 mRNA expression levels and cell viability.

In particular, MCF10CA1a.cl1 cells, MDA-MB231 cells, and the cells produced according to Example 3-1 were seeded in 96-wells at a density of 3,000 cells/well and incubated for 24 hours. The culture solution was removed and a culture solution containing paclitaxel or docetaxel was added to each well to a final volume of 100 μl/well. After incubation for 72 hours, the culture solution was removed from the culture, and 0.5 mg/ml of 3-(4,5-dimethylthiazol-2-yl)-2,5-diphenyltetrazolium bromide (MTT) was added to each well, followed by incubation for 2 hours. Media containing an MTT solution was carefully removed, and 200 μl of dimethyl sulfoxide was added to each well, followed by incubation at room temperature for 5 minutes. Absorbance at 580 nm was measured using a 96-well microplate detector (Molecular Devices).

In addition, total RNA was extracted from GFP virus-infected and Smad3 EPSM virus-infected cells using a TRIzol reagent (Invitrogen) in accordance with the manufacturer's protocol, and the extracted RNA was converted into cDNA using M-MLV reverse transcriptase (Promega). The cDNA was synthesized and subjected to RT-PCR using a specific primer pair by using an AccuPower™ PCR PreMix kit (Bioneer Co.). mRNAs of various genes were measured in triplicate and normalized to GAPDH.

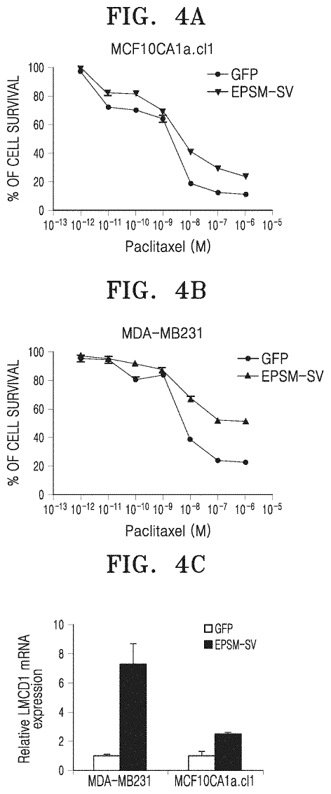

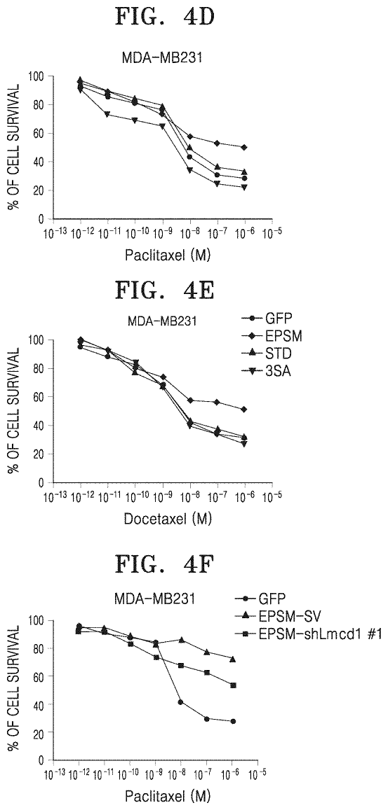

As a result, as can be seen from FIGS. 4A, 4B, 4D, and 4E, cell viability was increased compared to a control when Smad3 linker phosphorylation was blocked (EPSM), and the increase in cell viability was significant only when Smad3 linker phosphorylation was blocked (especially EPSM) compared to other phosphorylation sites, from which it was confirmed that paclitaxel resistance was induced. Along with an LMCD1 increase in paclitaxel-resistant cells, as can be seen from FIG. 4C, it was confirmed that an LMCD1 mRNA expression level was increased when Smad3 linker phosphorylation was blocked. This phenomenon indicates that, when LMCD1 is depleted in Smad3 EPSM adenovirus-infected cells (EPSM-SV-shLmcd1 #1), susceptibility to paclitaxel is restored, and thus LMCD1 expression is increased through inhibition of Smad3 linker phosphorylation, thus exhibiting a paclitaxel resistance acquisition phenomenon.

Thus, it can be seen that Smad3 phosphorylation inhibition (particularly, Smad3 linker phosphorylation inhibition) occurs along with LMCD1 overexpression in taxane-based drug-resistant cells.

### Example 4: Increasing Metastasis According to LMCD1 Overexpression

4-1. Metastasis Evaluation of Breast Cancer Cells by LMCD1 Overexpression

To evaluate whether metastasis of breast cancer cells is increased when LMCD1 is overexpressed, wound healing assay and migration and invasion assays were carried out.

In particular, first, for wound healing assay, MCF10CA1a.cl1 cells were seeded in a 6-well plate and incubated for 24 hours until all cultures reached a confluency suitable for use in experiments. A linear scratch was formed with the tip of a pipette, and the cells were observed and images thereof were acquired using a BX43 Clinical (1×51 Inverted) microscope (Olympus).

In addition, for the migration and invasion assays, dissociated MCF10CA1a.cl1 cells (5×10⁴ cells for the migration assay and 1×10⁵ cells for the invasion assay) were plated on upper wells of Transwell and Matrigel invasion chambers (BD Biosciences). For the invasion assay, the upper chambers included DMEM containing 0.1% FBS, and lower chambers included DMEM containing 10% FBS. After incubation for 24 hours, cells migrated through a membrane were fixed with 70% ethanol, and cells having not invaded and migrated were removed with a cotton swab, followed by staining with 0.05% crystal violet. For quantification, the cells were imaged using a microscope in four random fields and quantified using Image J software. In addition, for an LMCD1-depleted cell line, lentivirus for LMCD1 depletion was produced in the same manner as in Example 2-2 to infect the cell line.

In addition, total RNA was extracted from LMCD1-overexpressing MCF10CA1a.cl1 cells using a TRIzol reagent (Invitrogen) in accordance with the manufacturer's protocol, and the extracted RNA was converted into cDNA using M-MLV reverse transcriptase (Promega). The cDNA was synthesized and subjected to RT-PCR using a specific primer pair by using an AccuPower™ PCR PreMix kit (Bioneer Co.). mRNAs of various genes were measured in triplicate and normalized to GAPDH.

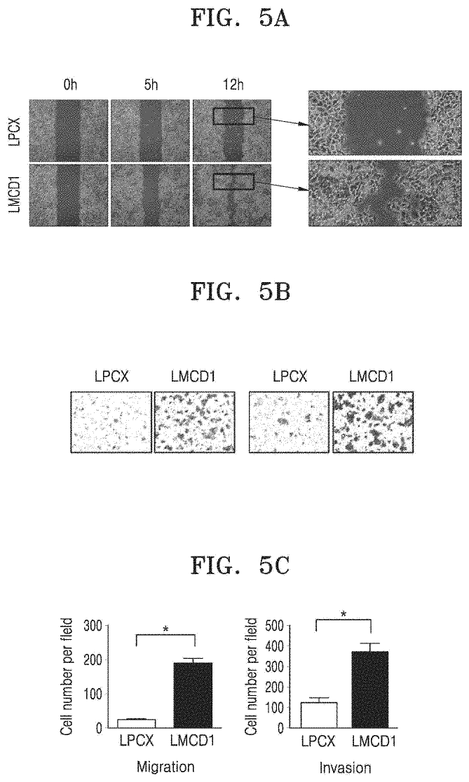

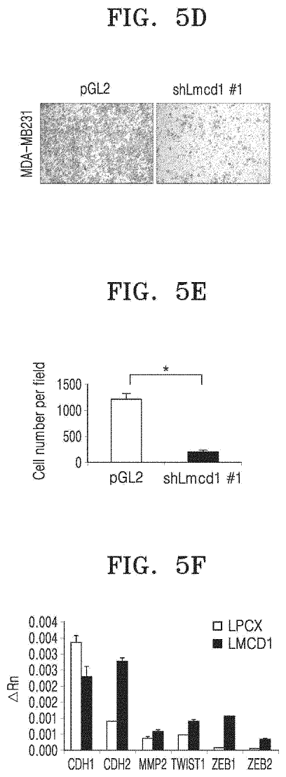

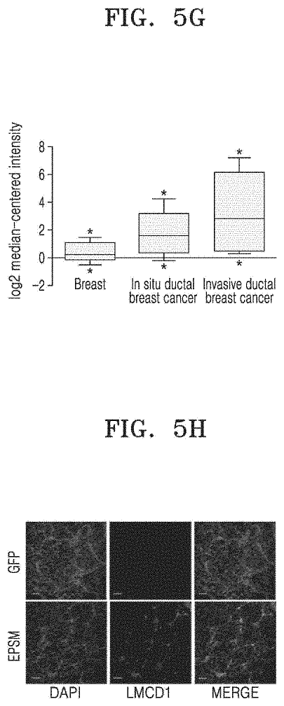

As a result, as illustrated in FIG. 5A, cell migration was increased in the case of LMCD1 overexpression, and as illustrated in FIGS. 5B-5C, invasion was also increased, and when LMCD1 expression was inhibited, migration and invasion were reduced again (see FIGS. 5D-5E5C). In addition, as illustrated in FIG. 5F, mRNA expression of EMT marker genes were increased by LMCD1 overexpression. Since the MCF10CA1a.cl1 cells used in these examples is known as a malignant breast cancer cell line with high metastasis, it can be seen from the above results that LMCD1 is involved in metastasis of breast cancer cells such as MCF10CA1a.cl1 cells, and thus characteristics of breast cancer cells, stages of breast cancer progression, post-treatment prognosis observation, or the presence or absence of metastasis and recurrence after complete cure may be monitored by measuring an LMCD1 expression level in a sample of a subject.

4-2. Clinical Statistical Evaluation of Presence or Absence of LMCD1 Overexpression in Metastatic Breast Cancer

To clinically evaluate whether LMCD1 overexpression occurs in metastatic breast cancer, public data analysis was carried out. In particular, LMCD1 expression levels of breast cancer and other types of cancer were analyzed using Oncomine Compendium of Expression Array data (https://www.oncomine.org). The P value for the analytical values was set to be 0.05.

As a result, it was also confirmed clinically that, when human breast cancer was metastatic (invasive), LMCD1 expression was further increased (see FIG. 5G).

4-3. Evaluation of LMCD1 in Smad3 Linker Phosphorylation Inhibition-Induced Lung Metastasis in Breast Cancer

To evaluate whether LMCD1 protein was present in metastasis acquisition by Smad3 linker phosphorylation inhibition, immunofluorescence staining analysis was performed.

In particular, 5×10⁵ MCF10CA1a.cl1 cells infected with Smad3 EPSM adenovirus were injected into the tail veins of female NOD/SCID mice obtained from Orient Bio Inc. (Seongnam, S. Korea). 3 weeks after injection, the mice were examined by autopsy to see if there was metastasis in internal organs of each mouse. Microscopic quantification of metastasis was performed on lung-transverse sections. Thereafter, paraffin blocks of lung into which GFP and Smad3 EPSM-infected MCF10CA1a.cl1 cells injected via the tail veins of NOS/SCID mice metastasized were prepared and cut into 4 μm-thick sections, and paraffin was removed therefrom in xylene and alcohol. After blocking endogenous peroxidase activity and a retrieving antigen, the sections were blocked with PBS containing 5% BSA at 37° C. for 1 hour. The sections were incubated with anti-LMCD1 (Abcam) overnight at 4° C. Incubation was performed at 1:500 for 1 hour using Alexa Fluor 488-conjugated goat anti-rabbit IgG as a secondary antibody. The sections were placed in a culture solution containing DAPI (Vector Laboratories) and evaluated using a confocal laser scanning microscope (LSM-510; Carl Zeiss).

As a result, as illustrated in FIG. 5H, it was confirmed that LMCD1 expression was significantly increased in the vicinity of lung metastasized by the Smad3 EPSM-infected MCF10ca1a.cl1 cells. It is also evident that, in the case of metastatic breast cancer, Smad3 linker phosphorylation inhibition and LMCD1 overexpression occur simultaneously.

### Example 5: Analysis of Expression Sub-Factors by LMCD1 Overexpression

5-1. Increase in Expression of Stemness Markers by LMCD1 Overexpression

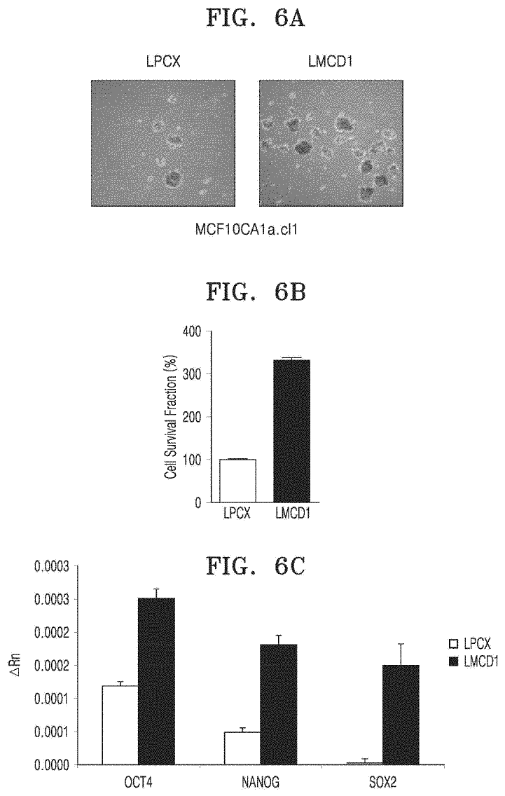

Referring to FIGS. 6A-6B, through analysis of mammosphere formation enabling observation of cancer cells having cancer stem cell-like characteristics, it was observed that cancer stemness of MCF10CA1a.cl1 cells was significantly increased when LMCD1 was overexpressed. Then, it was evaluated in terms of mRNA whether cancer stemness markers can be selected as factors capable of functioning as assay sub-markers for evaluating characteristics (e.g., metastasis, tumor formability, and the like) of breast cancer cells and the presence or absence of resistance to a specific drug, as well as LMCD1 overexpression.

In particular, MCF10CA1a.cl1 cells were maintained in a DMEM culture solution containing 10% fetal bovine serum and 1% penicillin/streptomycin (WeIGENE) in a CO₂ incubator at 37° C.

LMCD1 overexpression was performed in the same manner as described above.

Total RNA was extracted from the control and LMCD1-overexpressing cells using a TRIzol reagent (Invitrogen) in accordance with the manufacturer's protocol. The extracted RNA was converted into cDNA using M-MLV reverse transcriptase (Promega), and the cDNA was synthesized, followed by RT-PCR using a pair of primers specific to Oct4, Nanog, and Sox2 by using an AccuPower™ PCR PreMix kit (Bioneer Co.). mRNAs of various genes were measured in triplicate and normalized to GAPDH.

As a result, as illustrated in FIG. 6C, it was confirmed that the expression of Oct4, Nanog, and Sox2, which are cancer stemness markers, was significantly increased according to LMCD1 overexpression. From the above result, it is confirmed that the cancer stemness markers are able to function as assay sub-markers for evaluating characteristics of breast cancer cells and the presence or absence of resistance to a specific drug thereof, along with LMCD1 overexpression.

### Example 6: Response to TGF-β Treatment on Metastasis or Resistance to Taxanes and Smad3 Phosphorylation Activity

6-1. Analysis of Change in Expression Level of Intracellular Factor According to TGF-β Treatment and Smad3 Linker Phosphorylation Inhibition

Microarray assay was performed to investigate a factor exhibiting an in vivo change in expression level in accordance with TGF-β treatment and Smad3 linker phosphorylation inhibition.

In particular, MCF10CA1a.cl1 cells were infected with GFP, Smad3 wild-type, or Smad3 EPSM adenovirus for 48 hours, and then treated with TGF-β (5 ng/ml) and incubated for 24 hours, followed by DNA microarray assay.

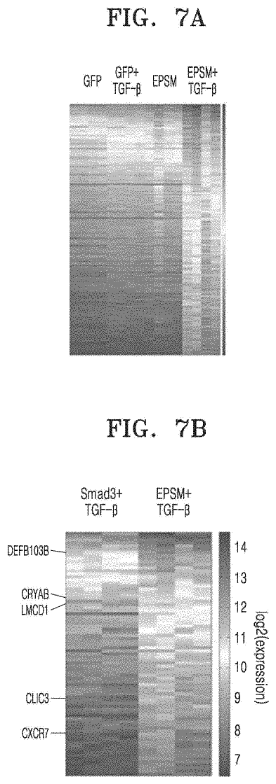

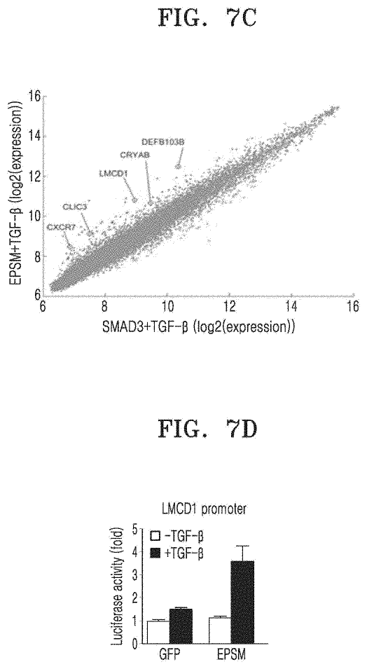

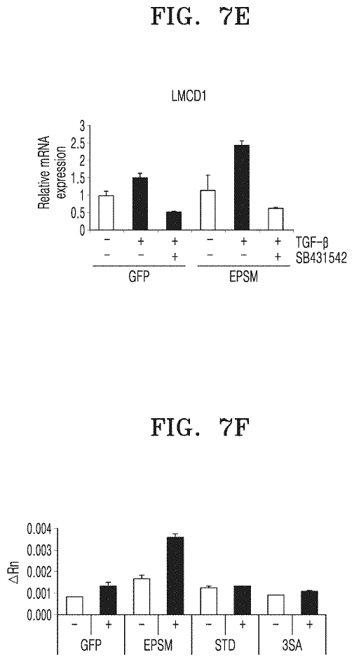

As a result, as illustrated in FIGS. 7A-7C, it was confirmed that LMCD1 was overexpressed in a sample treated with TGF-β in which Smad3 linker phosphorylation was inhibited.

6-2. Analysis of Change in Expression Level of LMCD1 According to TGF-β Treatment and Smad3 Linker Phosphorylation Inhibition

To confirm whether LMCD1 is induced by TGF-β treatment and Smad3 linker phosphorylation inhibition in the microarray analysis of Example 6-1, LMCD1 promoter analysis and mRNA change measurement were performed.

In particular, for the LMCD1 promoter analysis, MCF10CA1a.cl1 cells were infected with GFP and Smad3. EPSM adenovirus for 2 days, and then transiently transfected with an Lmcd1 promoter using FuGENE HD (Promega). 24 hours before harvesting, the cells were stimulated with 3 ng/ml of TGF-β. Luciferase activity was analyzed using a Luciferase Assay System kit (Promega) in accordance with the manufacturer's protocol. All assays were performed in triplicate, and all values were normalized to transfection efficiency as determined by β-galactosidase activity.

For the measurement of LMCD1 mRNA, MDA-MB231 cells were infected with GFP and Smad3. EPSM adenovirus for 2 days, and then 2 hours before TGF-β treatment, the cells were pre-treated with 10 μM of SB431542 and treated with 5 ng/ml of TGF-β, followed by incubation for 24 hours. Total RNA was extracted from the cells using a TRIzol reagent (Invitrogen) in accordance with the manufacturer's protocol. The extracted RNA was converted into cDNA using M-MLV reverse transcriptase (Promega). The cDNA was synthesized and subjected to RT-PCR using a specific primer pair by using an AccuPower™ PCR PreMix kit (Bioneer Co.). Gene mRNAs were normalized to GAPDH.

As a result, as illustrated in FIGS. 7D and 7E, it was confirmed that LMCD1 transcriptional activity and mRNA expression level were increased by TGF-β treatment and Smad3 linker phosphorylation inhibition.

6-3. Analysis of Change in LMCD1 Expression Level According to Smad3 Protein Phosphorylation Site

To confirm whether the LMCD1 overexpression phenomenon as described in Example 6-2 is induced by Smad3 linker phosphorylation inhibition, changes in LMCD1 mRNA in accordance with phosphorylation inhibition of each Smad3 phosphorylation site were measured.

In particular, GFP, Samd3 wild-type, C-terminal phosphorylation-inhibiting mutant (3SA), Smad3 linker phosphorylation-activated mutant (STD), and Smad3 linker phosphorylation-inhibiting mutant (EPSM) adenoviruses were supplied from Dr. Sushil G. Rane (NIDDK, NIH, Bethesda, Md.). To measure transduction efficiency, a GFP adenovirus was used as a control. The cells were infected with 75 to 100 virus particles/cell in a cell solution and stimulated with 5 ng/ml of TGF-β for 24 hours. Total RNA was extracted from the cells using a TRIzol reagent (Invitrogen) in accordance with the manufacturer's protocol. The extracted RNA was converted into cDNA using M-MLV reverse transcriptase (Promega). The cDNA was synthesized and subjected to RT-PCR using a specific primer pair by using an AccuPower™ PCR PreMix kit (Bioneer Co.). Gene mRNAs were normalized to GAPDH.

As a result, as illustrated in FIG. 7F, it was confirmed that, when Smad3 linker phosphorylation was specifically inhibited, LMCD1 overexpression by TGF-β treatment occurred.

Referring to Examples 1 to 5, when an LMCD1 intracellular expression level is increased, expression levels of cancer sternness- or metastasis-related factors are increased, and taxane-based drug resistance is increased. In addition, it was confirmed that the LMCD1 expression level was increased in metastatic cancer and taxane-based drug-resistant cancer, and it was actually observed that LMCD1 expression was also increased in metastatic breast cancer in clinical data analyses (see Example 4-2). Thus, it is confirmed that, when LMCD1 is overexpressed by stimulation of cells in a subject isolated from a subject by TGF-β, the subject may have metastasis or taxane-based drug resistance, and the inhibition of Smad3 protein phosphorylation (particularly, the inhibition of Smad3 linker phosphorylation) is also observed in the cells in a sample isolated from a subject.

### Example 7: Analysis of the Effect of LMCD1 Secretion on EMT and Drug Resistance in Breast Cancer

It has been reported that LMCD1 is mainly located in the nucleus and the cytoplasm. However, LMCD1 was identified for the first time by proteomics to be secreted to the extracellular space in the human aorta. To examine whether LMCD1 is secreted from breast cancer cells, we examined LMCD1 protein expression in the conditioned media of MCF10CA1a.cl1 cells.

In particular, for collection of the conditioned media, retrovirus-mediated LPCX (control) and LMCD1-overexpressing MCF10CA1a.cl1 cells were grown in 0.5% FBS-containing DMEM for 48 hours before harvest. The media was collected, centrifuged at 1,000 g for 10 minutes. The supernatant was carefully collected and filtered through Amicon Ultra-4 Centrigufal Filter Units (Millipore) according to the manufacturer's instruction. The biotinyated cell surface fractions were prepared by using Pierce Cell Surface Protein Isolation Kit (Thermo Scientific) according to the manufacturer's instruction. For preparation of total cell lysates, cells were lysed in a buffer containing 20 mM Hepes (pH 7.5), 150 mM NaCl, 1% Triton X-100, 10% glycerol, 5 mM EDTA, and protease inhibitor cocktail (Roche) on ice for 20 minutes. For the immunoprecipitation assay, cell extracts were incubated with the indicated primary antibodies overnight at 4° C. Antibody-bound proteins were precipitated with Dynabeads Protein G (Invitrogen) for 2 hours at 4° C., followed by washing four times with the same buffer in which cells were lysed. Samples were separated by SDS-PAGE, followed by electrotransfer to polyvinylidene difluoride membranes (Millipore). The membrane was blocked for 1 hour at room temperature (RT) and incubated overnight at 4° C. with the primary antibody. The primary antibodies used were as follows: LMCD1 (ab121788; Abcam), beta-actin (A5441; Sigma), Flag (F3165; Sigma). Horseradish peroxidase-conjugated anti-mouse/rabbit antibodies (Millipore, Temecula, Calif.) were used as secondary antibodies. The peroxidase reaction products were visualized by WESTZOL (Intron) and Amersham ECL™ Advance Western Blotting Detection Kit (GE Healthcare Life Sciences). All signals were detected by Amersham Imager 600 (GE Healthcare Life Sciences).

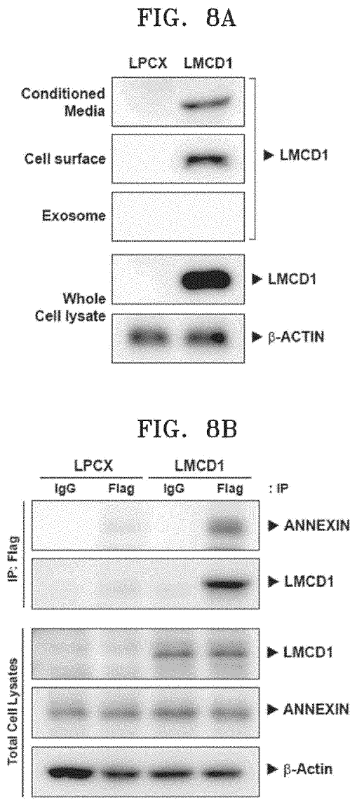

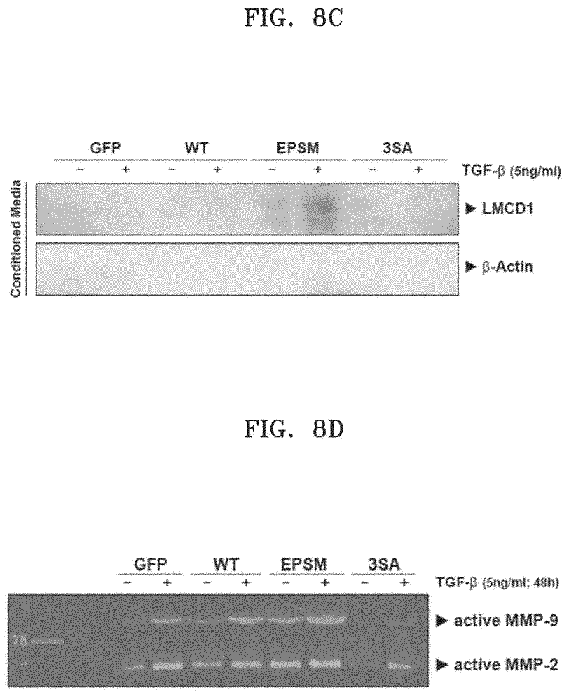

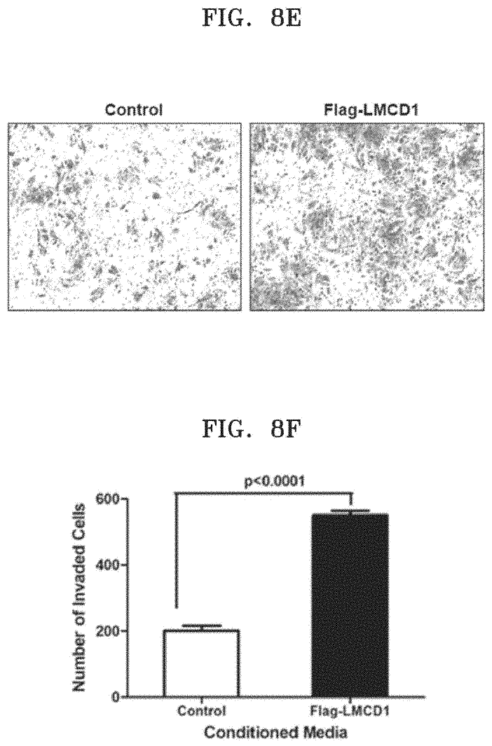

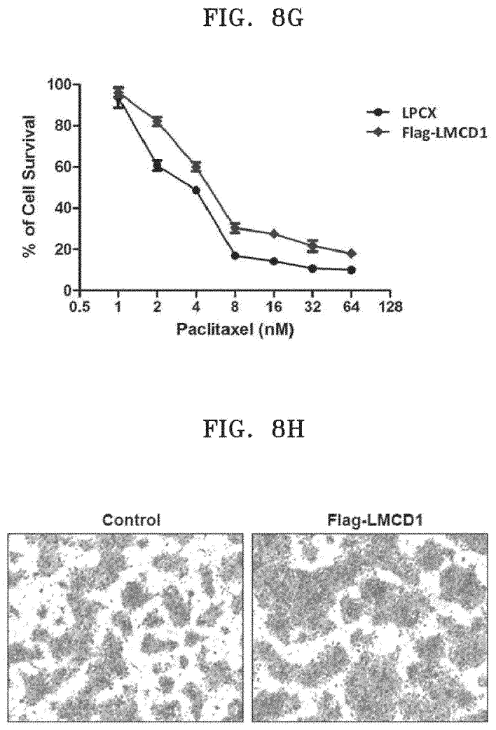

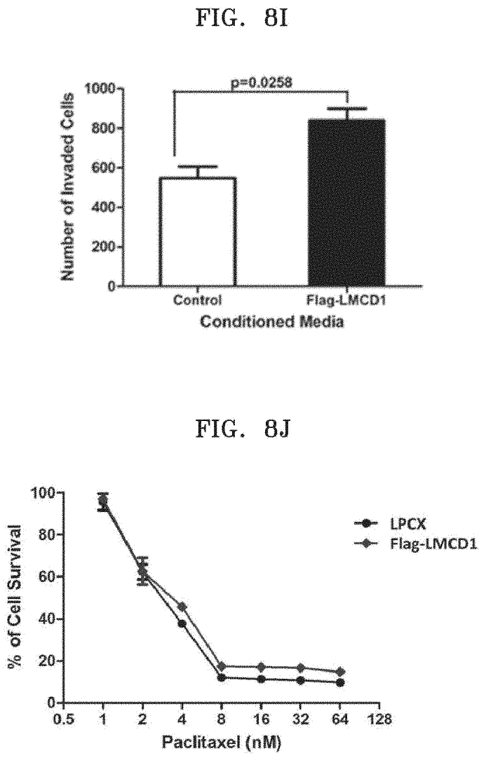

As a result, as illustrated in FIGS. 8A-8C, LMCD1 expression was confirmed not only in the conditioned media of LMCD1-overexpressing MCF10CA1a.cl1 cells, but also in the biotinylated cell surface fractions (FIG. 8A). The interaction between LMCD1 and Annexin II, which belongs to the annexin superfamily that binds to phospholipids and other proteins on the cell surface in a calcium-dependent manner, also supports that LMCD1 may be secreted to the extracellular space in a calcium-dependent manner (FIG. 8B). In addition, Smad3 EPSM adenovirus-mediated blockade of Smad3 linker phosphorylation induced LMCD1 secretion in the conditioned medium of MCF10CA1a.cl1 breast cancer cells (FIG. 8C).

Next, to confirm whether secreted LMCD1 induced EMT (epithelial-mesenchymal transition) and paclitaxel resistance of breast cancer cells, gelatin zymography assay, which detects the activity of matrix metalloproteinase (MMP-2 or MMP-9) that have been implicated in tumor cell invasion and metastasis was carried out.

In particular, MCF10CA1a.cl1 cells were placed in DMEM containing 0.5% FBS and infected with GFP, Smad3 WT, Smad3 EPSM, and Smad3 3SA adenoviruses for 48 hours in the absence or presence of TGF-β1 (5 ng/ml for 48 hours). The conditioned media were collected and concentrated as described in 1A. The 5× non-reducing sample buffer (4% SDS, 20% glycerol, 0.01% bromophenol blue, 125 mM Tris-HCl) was added to the concentrated conditioned media, and the complexes were ran on Novex 10% Zymogram Gels (ThermoFisher). The gel was washed with washing buffer (2.5% Triton X-100, 50 mM Tris-HCl, 5 mM CaCl₂), 1 μM ZnCl₂) at room temperature for 30 minutes, followed by incubation in a buffer (1% Triton X-100, 50 mM Tris-HCl, 5 mM CaCl₂), 1 μM ZnCl₂) for 24 hours at 37° C. Then, the gel was stained with Coomassie blue for 30 minutes to 1 hour at room temperature with agitation, followed by destaining in a solution (40% MeOH, 10% acetic acid) until bands were clearly seen.

For invasion assay, dissociated 1×10⁵ MCF10CA1a.cl1 cells were plated on upper wells of Matrigel invasion chambers (BD Biosciences). The concentrated conditioned media acquired from LPCX and LMCD1-overexpressing MCF10CA1a.cl1 cells were added to the upper chambers. The lower chambers included DMEM containing 10% FBS. After incubation for 24 hours, cells migrated through a membrane were fixed with 70% ethanol, and cells having not invaded and migrated were removed with a cotton swab, followed by staining with 0.05% crystal violet. For quantification, the cells were imaged using a microscope in four random fields and quantified using Image J software.

For MTT assay to examine resistance to paclitaxel, the cells were seeded in 96 wells at a density of 3,000 cells per well and incubated for 24 hours. The culture solution was removed, and a culture solution containing the concentrated conditioned media acquired from LPCX and LMCD1-overexpressing MCF10CA1a.cl1 cells was added to each well to a final volume of 100 μl/well. After incubation for 72 hours, the culture solution was removed from the culture, and 0.5 mg/ml of 3-(4,5-dimethylthiazol-2-yl)-2,5-diphenyltetrazolium bromide (MTT) was added to each well, followed by incubation for 2 hours. PBS containing an MTT solution was carefully removed, and 200 μl of dimethyl sulfoxide was added to each well, followed by incubation at room temperature for 5 minutes. Absorbance at 580 nm was measured using a 96-well microplate detector (Molecular Devices).

As a result, as illustrated in FIG. 8D, MMP-2 and MMP-9 activity was significantly increased in the conditioned media of EPSM-infected MCF10CA1a.cl1 cells (which Smad3 linker phosphorylation is inactive) even in the absence of TGF-β. It can be seen from the above results that the secreted LMCD1, which is induced by TGF-β and Smad3 linker phosphorylation inhibition, may enhance the metastatic potential of breast cancer cells.

In addition, as illustrated in FIGS. 8E-8J, it was confirmed that resistance to paclitaxel and invasiveness was significantly increased, when treatment of the conditioned media of LMCD1-overexpressing MCF10CA1a.cl1 cells to the wild-type MCF10CAT1k.cl2 (M-II), which forms benign hyperplastic lesions, and MCF10CA1a.cl1 cells. Taken together, these data indicate that secreted LMCD1 is associated with increased EMT and acquired drug resistance of breast cancer cells.

### Example 8: Analysis of LMCD1 Secretion in the Serums of Breast Cancer Patients

The measured LMCD1 expression level in the serum of breast cancer patients was compared with that of healthy volunteers. In particular, the serums of healthy volunteers and breast cancer patients of different subtypes were provided by the Gangnam Severance Hospital, Yonsei University College of Medicine (Seoul, S. Korea). LMCD1 protein was detected by using LMCD1 Elisa Kit (MyBiosource) according to the manufacturer's instruction.

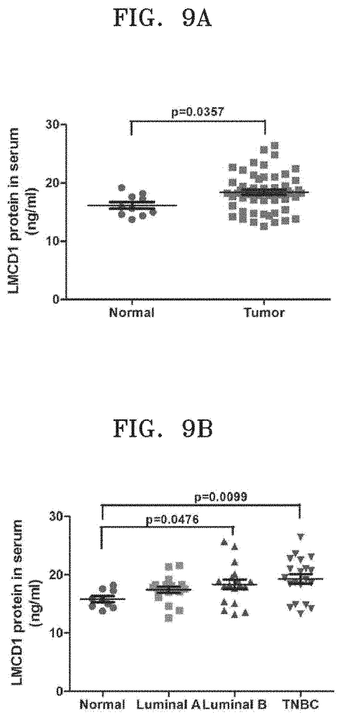

As a result, as illustrated in FIGS. 9A and 9B, increased LMCD1 protein level was observed in the serums of breast cancer patients compared to those of healthy volunteers (FIG. 9A). Moreover, higher level of LMCD1 was found in the serums of the patients with luminal B and triple negative breast cancer (TNBC) subtype, which are more invasive and frequently involve metastasis and drug resistance (FIG. 9B). These findings suggest that measurement of LMCD1 level in the serum may serve as a marker that predicts metastatic potential or the subtype of breast cancer.

1. A method of diagnosing metastatic cancer or taxane-based drug-resistant cancer, the method comprising:
measuring an LMCD1 expression level from a complex formed by bringing, into contact with a sample isolated from a subject, an antibody, peptide, protein, or combination thereof that specifically binds to an LMCD1 protein or a fragment thereof; or a probe, primer, nucleotide, or combination thereof that specifically binds to a nucleotide sequence encoding the LMCD1 protein; and
comparing the measured LMCD1 expression level of the sample with a measured LMCD1 expression level of a control.

2. The method of claim 1, wherein the LMCD1 protein comprises any one sequence selected from SEQ ID NOS: 1 to 4, and the nucleotide sequence encoding the LMCD1 protein comprises any one sequence selected from SEQ ID NOS: 5 to 8.

3. The method of claim 1, further comprising, before the measuring of the LMCD1 expression level, bringing TGF-β into contact with the sample isolated from a subject.

4. The method of claim 1, further comprising measuring a degree of phosphorylation of a Smad3 linker or its carboxy terminus from a complex formed by bringing, into contact with the sample isolated from a subject, an antibody, peptide, protein, or combination thereof that specifically binds to a phosphorylated Smad3 linker, its carboxy-terminus, or a fragment thereof.

5. The method of claim 4, wherein the Smad3 linker or its carboxy-terminus comprise amino acid residues 143-230 or amino acid residues 422-425 in an amino acid sequence of SEQ ID NO: 9.

6. The method of claim 4, wherein the phosphorylation comprises phosphorylation of at least one selected from the group consisting of amino acid residues 179, 204, 208, 213, 422, 423, and 425 in an amino acid sequence of the Smad3 linker or its carboxy terminus.

7. The method of claim 3, further comprising measuring a degree of phosphorylation of a Smad3 linker or its carboxy terminus from a complex formed by bringing, into contact with the sample isolated from a subject, an antibody, peptide, protein, or combination thereof that specifically binds to a phosphorylated Smad3 linker, its carboxy-terminus, or a fragment thereof.

8. The method of claim 7, wherein the Smad3 linker or its carboxy-terminus comprise amino acid residues 143-230 or amino acid residues 422-425 in an amino acid sequence of SEQ ID NO: 9.

9. The method of claim 7, wherein the phosphorylation comprises phosphorylation of at least one selected from the group consisting of amino acid residues 179, 204, 208, 213, 422, 423, and 425 in an amino acid sequence of the Smad3 linker or its carboxy terminus.

10. The method of claim 1, further comprising measuring an expression level of a cancer stemness marker from a complex formed by bringing, into contact with the sample isolated from a subject, an antibody, peptide, protein, or combination thereof that specifically binds to a cancer stemness marker protein or a fragment thereof; or a probe, primer, nucleotide, or combination thereof that specifically binds to a nucleotide sequence encoding the cancer stemness marker protein, wherein the cancer stemness marker is Oct4, Nanog, Sox2, CD44, CD24, ALDH1, CD326 (EpCAM), or a combination thereof.

11. The method of claim 1, wherein the measuring of the LMCD1 expression level is performed using at least one method selected from RT-PCR, RNase protection assay (RPA), Northern blotting, and a DNA CHIP.

12. The method of claim 1, wherein the measuring of the LMCD1 expression level is performed using at least one method selected from Western blotting, ELISA, radioimmunoassay, radioimmunodiffusion, Ouchterlony immunodiffusion, rocket immunoelectrophoresis, immunohistostaining, immunoprecipitation assay, complement fixation assay, FACS, and a protein chip.

13. The method of claim 1, wherein the cancer is breast cancer.

14. The method of claim 13, wherein the cancer is metastatic breast cancer.

15. The method of claim 1, wherein the sample isolated from a subject is a cell, an organ, a cell lysate, blood, serum, plasma, lymph fluid, extracellular fluid, body fluid, urine, feces, tissue, bone marrow, saliva, sputum, cerebrospinal fluid, or a combination thereof.

16. The method of claim 1, further comprising administrating anti-cancer drug except taxane-based drug to the subject diagnosed with metastatic cancer or taxane-based drug-resistant cancer.

17. The method of claim 16, wherein the anti-cancer drug except taxane-based drug treatment is Anthracyclines, Platinum-based drugs, Vinorelbine, Capecitabine, Gemcitabine, Ixabepilone, Eribulin or combination thereof.

18. The method of claim 17, wherein the anti-cancer drug except taxane-based drug treatment is platinum-based drugs.
